(19) United States
(12) Patent Application Publication
(10) Pub. No.: US 2018/0153984 A1
(43) Pub. Date: Jun. 7, 2018

(54) ADJUVANT PARTICLES COMPRISING ADENOSINE RECEPTOR ANTAGONISTS

(71) Applicants: The Regents of the University of California, Oakland, CA (US); Therinject LLC, San Diego, CA (US)

(72) Inventors: Peter ERNST, Solana Beach, CA (US); Steven JOSEPHS, San Diego, CA (US)

(21) Appl. No.: 15/569,928

(22) PCT Filed: Apr. 29, 2016
(86) PCT No.: PCT/US2016/030056

§ 371 Date: Oct. 27, 2017

Related U.S. Application Data
(60) Provisional application No. 62/154,870, filed on Apr. 30, 2015.

(51) Int. Cl. A61K 39/39; A61K 31/519; A61K 9/16; A61K 45/06; A61P 31/04; A61P 33/00; A61K 39/00

(52) U.S. Cl. CPC A61K 39/39; A61K 31/519; A61K 9/1647; A61K 9/1652; A61K 45/06; A61K 2039/55555; A61P 33/00; A61K 39/0011; A61K 2039/55583; A61K 2039/55511; A61P 31/04

(57) ABSTRACT

This document relates to polymeric particles for enhancing the immune response, compositions comprising the polymeric particles, and methods of use thereof. The polymeric particles include a permeation enhancer, an adenosine receptor antagonist, and optionally a biodegradable polymer, wherein the polymeric particles are useful as adjuvant compositions.

### CLAIM OF PRIORITY

This application claims the benefit of U.S. Provisional Patent Application Ser. No. 62/154,870, filed on Apr. 30, 2015, the entire contents of which are hereby incorporated by reference.

### FEDERALLY SPONSORED RESEARCH OR DEVELOPMENT

This invention was made with government support under Grant No. AI105916 awarded by the National Institute of Health. The government has certain rights in the invention.

### TECHNICAL FIELD

This document relates to methods and materials for inducing and/or enhancing an immune response.

### BACKGROUND

Adjuvants are materials that can be used either for the development of vaccines having increased antigenicity or for therapeutic and preventive purposes by enhancing non-specific immune responses to antigens. In addition, the adjuvants can be used to enhance immune responses, particularly in immunologically immature or senescent persons, in order to enhance the induction of mucous immunity.

Over half of the world's population is infected with Helicobacter pylori (Ernst et al., The translation of Helicobacter pylori basic research to patient care. Gastroenterology. 2006; 130(1):188-206; Fox et al., Inflammation, atrophy, and gastric cancer. Journal of Clinical Investigation. 2007; 117(1):60-9; Polk D B, Peek R M, Jr. Helicobacter pylori: gastric cancer and beyond. Nat Rev Cancer. 2010; 10(6):403-14; and Graham et al., Helicobacter pylori treatment in the era of increasing antibiotic resistance. Gut. 2010; 59(8):1143-53; incorporated herein in their entirety). Most individuals get infected in childhood and infection usually persists for life (Schwarz et al., Horizontal versus familial transmission of Helicobacter pylori. PLoS Pathog. 2008; 4(10):e1000180; and Ashorn et al.,” Seroepidemiological study of Helicobacter pylori infection in infancy.” Archives of Disease in Childhood Fetal & Neonatal Edition. 1996; 74:F141-2; incorporated herein in their entirety). It is the persistence of this infection that leads to the sequence of events culminating in gastroduodenal ulceration, gastric cancer and lymphoma (Emst et al., The translation of Helicobacter pylori basic research to patient care. Gastroenterology. 2006; 130(1):188-206; Fox et al., Inflammation, atrophy, and gastric cancer. Journal of Clinical Investigation. 2007; 117(1):60-9; Polk et al., Helicobacter pylori: gastric cancer and beyond. Nat Rev Cancer. 2010; 10(6):403-14; Graham et al., Helicobacter pylori treatment in the era of increasing antibiotic resistance. Gut. 2010; 59(8): 1143-53; Correa P. Helicobacter pylori and gastric carcinogenesis. American Journal of Surgical Pathology. 1995; 19:S37-S43; Uemura et al., Helicobacter pylori infection and the development of gastric cancer. New England Journal of Medicine. 2001; 345(11):784-9; and McColl K E. Clinical practice. Helicobacter pylori infection. N Engl J Med. 2010; 362(17):1597-604; incorporated herein in their entirety). The WHO describes gastric cancer as the second leading cause of cancer mortality in the world. Even in the United States, it remains a leading cause of cancer death particularly in the groups lacking healthcare including minorities (Latino, African-American), immigrants from Asia and Latin America and migrant workers from poorer countries. While children in the United States fortunate to be in the middle or higher economic classes have a lifetime incidence of approximately 10%, poorer communities, such as African-Americans in the South, have 80% prevalence of infection (Epplein et al., Race, African ancestry, and Helicobacter pylori infection in a low-income United States population. Cancer Epidemiol Biomarkers Prev. 2011; 20(5):826-34; incorporated herein in its entirety). Thus, it is predominantly a disease of the poor and under-represented.

Epidemiological studies have failed to identify simple interventions that prevent infection. Antibiotic treatment is only 85% effective and recommended primarily for recurrent ulcers and as an adjunctive therapy for patients with early stage gastric maltomas (Emst et al., The translation of Helicobacter pylori basic research to patient care. Gastroenterology. 2006; 130(1):188-206; Graham et al., Helicobacter pylori treatment in the era of increasing antibiotic resistance. Gut. 2010; 59(8):1143-53; and Peura D A. Treatment of Helicobacter pylori infection. In: Wolfe M M, editor. Therapy of Digestive Disorders. Philadelphia: Elsevier Inc.; 2006. p. 277-90; incorporated herein in their entirety). Thus, novel interventional strategies are needed to avoid the clinical consequences of chronic inflammation induced by H. pylori.

### SUMMARY

The present disclosure is based, at least in part, on the development of polymeric particles comprising a permeation enhancer and an adenosine receptor antagonist, and optionally a biodegradable polymer, that have several advantages including, for example, for use as an adjuvant composition. In some aspects, the particle is a nanoparticle or microparticle.

In some aspects, the disclosure provides a particle that includes a permeation enhancer and an adenosine receptor antagonist. In some embodiments of all aspects, the particle is a nanoparticle or microparticle. In some cases, the particle further includes a biodegradable polymer.

In some embodiments of all aspects, the particle also includes an antigen. In some cases, the antigen is a bacterial antigen, a viral antigen or a tumor antigen. In some cases, the antigen is an H. pylori antigen. In some instances, the antigen is disposed on or presented on the surface of the particle. In some instances, the antigen is disposed throughout the particle or mixed within the particle.

In some embodiments of all aspects, the particle also includes a therapeutic agent such as an antimicrobial agent, an antibiotic agent, an anti-fungal agent, an anti-cancer agent, an anti-tumor agent, a signaling protein, a small molecule drug, a nucleic acid composition, a peptide therapeutic and/or an antibody.

In some embodiments of all aspects, the adenosine receptor antagonist is selected from the group consisting of caffeine, theophylline, 8-phenyl theophylline, SCH58261, istradefylline, pyrazolo[4,3-e]-1,2,4-triazolo[1,5-c]pyrimidines or substituted derivatives thereof (e.g., methoxy biaryl or quinoline substitutions), SCH412348, SCH420814, fused heterocyclic pyrazolo[4,3-e]-1,2,4-triazolo[1,5-c]pyrimidines or substituted derivatives thereof (e.g., tetrahydyroisoquinoline or azaisoquinoline derivatives), aryl piperazine substituted 3H-[1,2,4]-triazolo[5,1-i]purin-5-amines, arylindenopyrimidines, arylindenopyrimidines or substituted derivatives thereof, pyrazolo[4,3-e]-1,2,4-trizolo[4,3-c]pyrimidon-3-one and thiazolotriazolopyrimidines, 1,2,4-triazolo[1,5-c]pyrimidines or substituted derivatives thereof, purinones or substituted derivatives thereof, thieno[3,2-d]pyrimidines, pyrazolo[3,4-d]pyrimidines, and 6-arylpurines, benzyl substituted triazolo[4,5-d]pyrimidines, triazolo-9H-purines, aminomethyl substituted thieno[2,3-d]pyrimidines, 2-Aminoimidazopyridines, 4-morpholino-benzothiazoles or substituted derivatives thereof, 4-Aryl and 4-morpholino substituted benzofurans, pyridone substituted pyrazines, heterocyclic substituted 2-amino-thiazoles, trisubstituted pyrimidines, piperazine substituted pyrimidine acetamides, acylaminopyrimidines, pyrimidine, pyridine, or triazine carboxamides, mixtures or combinations thereof, and pharmaceutically acceptable salts thereof.

In some embodiments of all aspects, the permeation enhancer is selected from the group consisting of chitosan material, a fatty acid, a bile salt, a salt of fusidic acid, a polyoxyethylenesorbitan, a sodium lauryl sulfate, polyoxyethylene-9-lauryl ether (LAURETH™-9), EDTA, citric acid, a salicylate, a caprylic glyceride, a capric glyceride, sodium caprylate, sodium caprate, sodium laurate, sodium glycyrrhetinate, dipotassium glycyrrhizinate, glycyrrhetinic acid hydrogen succinate, a disodium salt, a nacylcamitine, a cyclodextrin, a phospholipid, and mixtures or combinations thereof.

In some embodiments of all aspects, the biodegradable polymer is selected from the group consisting of a polyester, a lactic acid polymer, copolymers of lactic acid and of glycolic acid (e.g., poly lactic acid (PLA), poly glycolic acid (PGA), or poly (lactic-co-gly colic acid) (PLGA”)), poly-ε-caprolactone (PCL), poly(anhydrides), poly(amides), poly(urethanes), poly(carbonates), poly(acetals), poly(ortho-esters), poly(glycolide-co-trimethylene carbonate), poly(dioxanone), poly(phosphoesters), poly(phosphazenes), poly(cyanoacrylate), poly(ethylene oxide), poly(propylene oxide), poly(N-isopropylacrylamide) (PNIPAAm), poly(2-(diethylamino)ethyl methacrylate) (PDEAEMA), poly(2-aminoethyl methacrylate) (PAEMA), 2 (dimethylamino)ethyl methacrylate (DMAEMA), poly(ethylene glycol) (PEG), N-(2-hydroxypropyl)methacrylamide (HPMA), poly(β-benzyl-1-aspartate) (PBLA), poly(hydroxybutyrate-co valerate), derivatives thereof, and mixtures or combinations thereof. In some instances the copolymers of lactic acid and of glycolic acid are selected from PLA, PGA, and PLGA.

In some embodiments, the particle also includes a dye. In some cases, the dye is selected from the group consisting of DiD dye, DiO dye, DiA dye, DiI dye, and DiR dye.

In some embodiments of all aspects, the particle has an average diameter of about 0.5 nm to about 80 m. In some instances, the particle has an average diameter of about 0.5 nm to about 1,000 nm. In some instances, the particle has an average diameter of about 1 μm to about 80 μm.

In some cases, the biodegradable polymer is PLGA, the permeation enhancer is chitosan, and the adenosine receptor antagonist is selected from the group consisting of SCH58261 and theophylline.

In some embodiments of all aspects, the particle also includes a targeting moiety. In some cases, the targeting moiety is selected from the group consisting of a tumor-targeting moiety, a viral-specific moiety, a bacteria-specific moiety, and a cell-targeting moiety. In some cases, the targeting moiety is a cell-targeting moiety and is selected from the group consisting of a phagocytic cell-targeting moiety, a natural killer cell-targeting moiety, a T-cell targeting moiety, a B-cell targeting moiety, a glial cell targeting moiety, a myeloid cell targeting moiety, an epithelial cell targeting moiety, a macrophage-targeting moiety, a tumor cell-targeting moiety, and a dendritic cell-targeting moiety.

In another aspect, the disclosure provides a pharmaceutical composition comprising the particles described herein. In some embodiments of all aspects, the pharmaceutical composition further comprises an antigen. In some embodiments of all aspects, the composition is formulated for parenteral, intravenous, intradermal, subcutaneous, oral, inhalation, transdermal, transmucosal, rectal and intratumoral administration.

In another aspect, the disclosure provides an adjuvant composition comprising the particles described herein.

In another aspect, the disclosure provides a vaccine composition that includes a particle comprising a permeation enhancer and an adenosine receptor antagonist; and an antigen. In some embodiments, the particle also includes a biodegradable polymer. In some embodiments, the antigen is disposed on or presented on the surface of the particle. In some embodiments, the antigen is disposed throughout the particle or mixed throughout the particle. In some embodiments, the antigen is a H. pylori antigen.

In some embodiments of all aspects, the composition also includes a therapeutic agent selected from the group consisting of an antimicrobial agent, an antibiotic agent, an anti-fungal agent, an anti-cancer agent, an anti-tumor agent, a signaling protein, a small molecule drug, a nucleic acid composition, a peptide therapeutic and an antibody.

In some instances, the adenosine receptor antagonist is selected from the group consisting of caffeine, theophylline, 8-phenyl theophylline, SCH58261, istradefylline, pyrazolo[4,3-e]-1,2,4-triazolo[1,5-c]pyrimidines or substituted derivatives thereof (e.g., methoxy biaryl or quinoline substitutions), SCH412348, SCH420814, fused heterocyclic pyrazolo[4,3-e]-1,2,4-triazolo[1,5-c]pyrimidines or substituted derivatives thereof (e.g., tetrahydyroisoquinoline or azaisoquinoline derivatives), aryl piperazine substituted 3H-[1,2,4]-triazolo[5,1-i]purin-5-amines, arylindenopyrimidines, arylindenopyrimidines or substituted derivatives thereof, pyrazolo[4,3-e]-1,2,4-trizolo[4,3-c]pyrimidon-3-one and thiazolotriazolopyrimidines, 1,2,4-triazolo[1,5-c]pyrimidines or substituted derivatives thereof, purinones or substituted derivatives thereof, thieno[3,2-d]pyrimidines, pyrazolo[3,4-d]pyrimidines, and 6-arylpurines, benzyl substituted triazolo[4,5-d]pyrimidines, triazolo-9H-purines, aminomethyl substituted thieno[2,3-d]pyrimidines, 2-Aminoimidazopyridines, 4-morpholino-benzothiazoles or substituted derivatives thereof, 4-Aryl and 4-morpholino substituted benzofurans, pyridone substituted pyrazines, heterocyclic substituted 2-amino-thiazoles, trisubstituted pyrimidines, piperazine substituted pyrimidine acetamides, acylaminopyrimidines, pyrimidine, pyridine, or triazine carboxamides, and mixtures or and pharmaceutically acceptable salts thereof.

In some instances, the permeation enhancer is selected from the group consisting of chitosan material, a fatty acid, a bile salt, a salt of fusidic acid, a polyoxyethylenesorbitan, a sodium lauryl sulfate, polyoxyethylene-9-lauryl ether (LAURETH™-9), EDTA, citric acid, a salicylate, a caprylic glyceride, a capric glyceride, sodium caprylate, sodium caprate, sodium laurate, sodium glycyrrhetinate, dipotassium glycyrrhizinate, glycyrrhetinic acid hydrogen succinate, a disodium salt, a nacylcarnitine, a cyclodextrin, a phospholipid, and mixtures or combinations thereof.

In some cases, the biodegradable polymer is selected from the group consisting of a polyester, a lactic acid polymer, copolymers of lactic acid and of glycolic acid, poly-ε-caprolactone (PCL), poly(anhydrides), poly(amides), poly(urethanes), poly(carbonates), poly(acetals), poly(ortho-esters), poly(glycolide-co-trimethylene carbonate), poly(dioxanone), poly(phosphoesters), poly(phosphazenes), poly(cyanoacrylate), poly(ethylene oxide), poly(propylene oxide), poly(N-isopropylacrylamide) (PNIPAAm), poly(2-(diethylamino)ethyl methacrylate) (PDEAEMA), poly(2-aminoethyl methacrylate) (PAEMA), 2 (dimethylamino)ethyl methacrylate (DMAEMA), poly(ethylene glycol) (PEG), N-(2-hydroxypropyl)methacrylamide (HPMA), poly(β-benzyl-1-aspartate) (PBLA), poly(hydroxybutyrate-co valerate), derivatives thereof, and mixtures or combinations thereof. In some instances, the copolymers of lactic acid and of glycolic acid are selected from PLA, PGA, and PLGA.

In some embodiments, the particle further comprises a dye. In some cases, the dye is selected from the group consisting of DiD dye, DiO dye, DiA dye, DiI dye, and DiR dye.

In some embodiments of all aspects, the particle has an average diameter of about 0.5 nm to about 80 μm. In some instances, the particle has an average diameter of about 0.5 nm to about 1,000 nm. In some instances, the particle has an average diameter of about 1 μm to about 80 μm.

In some embodiments, the biodegradable polymer is PLGA, the permeation enhancer is chitosan, and the adenosine receptor antagonist is selected from the group consisting of SCH58261 and theophylline.

In some cases, the composition also includes a targeting moiety. In some cases, the targeting moiety is selected from the group consisting of a tumor-targeting moiety, a viral-specific moiety, a bacteria-specific moiety, and a cell-targeting moiety. In some cases, the targeting moiety is a cell-targeting moiety and is selected from the group consisting of a phagocytic cell-targeting moiety, a macrophage-targeting moiety and a dendritic cell-targeting moiety.

In some embodiments of all aspects, the composition is formulated for parenteral, intravenous, intradermal, subcutaneous, oral, inhalation, transdermal, transmucosal, rectal and intratumoral administration.

In another aspect, the disclosure provides a method of treating an infectious disease, comprising administering a therapeutically effective amount of the particles described herein. In another aspect, the disclosure provides a method of treating an infectious disease, comprising administering a therapeutically effective amount of a pharmaceutical composition described herein.

In another aspect, the disclosure provides a method of treating an infectious disease, comprising administering a therapeutically effective amount of a pharmaceutical composition comprising a particle described herein. In some embodiments the pharmaceutical composition further comprises an antigen. In some instances, the antigen is a bacterial antigen, a viral antigen or a tumor antigen. In some cases, the antigen is an H. pylori antigen. In some instances, the antigen is disposed on or presented on the surface of the particle. In some instances, the antigen is disposed throughout the particle or mixed within the particle. In some instances, the antigen is co-administered before, after, or concurrently with administration of the composition comprising the particles described herein.

In some embodiments of all aspects, the method further includes administering a therapeutically effective amount of an antimicrobial agent. In some cases, the antimicrobial agent is administered before, after or concurrently with the administering of the particle.

In another aspect, the disclosure provides a method of treating an infectious disease, comprising: identifying a subject having the infectious disease; and administering a therapeutically effective amount of a pharmaceutical composition described herein, wherein the pharmaceutical composition comprises an antigen selected from a bacterial antigen and a viral antigen. In some embodiments, the method further includes administering a therapeutically effective amount of an antimicrobial agent.

In another aspect, the disclosure provides a method of vaccinating a subject, comprising administering a therapeutically effective amount of a vaccine composition described herein. In some embodiments, the vaccine is administered prophylactically.

In another aspect, the disclosure provides a method of treating a tumor in a subject in need thereof, comprising administering to the subject a therapeutically effective amount of a pharmaceutical composition described herein. In some instances, the pharmaceutical composition comprises an anti-tumor antigen. In some cases, the method further includes administering a therapeutically effective amount of an anti-tumor agent. In some embodiments of all aspects the anti-tumor agent is administered before, after, or concurrently with the administering of the particle. In some cases, the administration is performed by intratumoral injection.

In another aspect, the disclosure provides a method of treating an infection in a subject in need thereof, comprising administering to the subject a therapeutically effective amount of a pharmaceutical composition comprising a particle described herein to the subject. In some cases, the method further comprises administering a therapeutically effective amount of an agent selected from the group consisting of an antibiotic, an anti-fungal, an anti-viral, and anti-parasitic agent. In some instances, the agent is administered before, after, or concurrently with the administering of the particle. In some cases, the infection is a persistent infection.

In another aspect, the disclosure provides a method of treating a H. pylori infection in a subject in need thereof, comprising administering to the subject a therapeutically effective amount of a pharmaceutical composition described herein to the subject. In some embodiments, the permeation enhancer is chitosan. In some cases, the adenosine receptor antagonist is SCH58261 or theophylline. In some embodiments, the pharmaceutical composition comprises an antigen and the antigen is an H. pylori antigen. In some cases, the pharmaceutical composition includes a biodegradable polymer, which is PLGA. In some cases, the subject has been diagnosed with an infection mediated by H. pylori. In some cases, the infection is a persistent infection.

In another aspect, the disclosure provides a method of enhancing an immune response to an antigen comprising: administering: (1) a particle comprising a biodegradable polymer; a permeation enhancer; and an adenosine receptor antagonist; and (2) an antigen. In some instances, the antigen is a H. pylori antigen. In some instances, the antigen is a tumor antigen. In some cases, the antigen is disposed on the surface of the particle. In some cases, the antigen is disposed throughout the particle.

In some embodiments, the adenosine receptor antagonist is selected from the group consisting of caffeine, theophylline, 8-phenyl theophylline, SCH58261, istradefylline, pyrazolo[4,3-e]-1,2,4-triazolo[1,5-c]pyrimidines or substituted derivatives thereof (e.g., methoxy biaryl or quinoline substitutions), SCH412348, SCH420814, fused heterocyclic pyrazolo[4,3-e]-1,2,4-triazolo[1,5-c]pyrimidines or substituted derivatives thereof (e.g., tetrahydyroisoquinoline or azaisoquinoline derivatives), aryl piperazine substituted 3H-[1,2,4]-triazolo[5,1-i]purin-5-amines, arylindenopyrimidines, arylindenopyrimidines or substituted derivatives thereof, pyrazolo[4,3-e]-1,2,4-trizolo[4,3-c]pyrimidon-3-one and thiazolotriazolopyrimidines, 1,2,4-triazolo[1,5-c]pyrimidines or substituted derivatives thereof, purinones or substituted derivatives thereof, thieno[3,2-d]pyrimidines, pyrazolo[3,4-d]pyrimidines, and 6-arylpurines, benzyl substituted triazolo[4,5-d]pyrimidines, triazolo-9H-purines, aminomethyl substituted thieno[2,3-d]pyrimidines, 2-Aminoimidazopyridines, 4-morpholino-benzothiazoles or substituted derivatives thereof, 4-Aryl and 4-morpholino substituted benzofurans, pyridone substituted pyrazines, heterocyclic substituted 2-amino-thiazoles, trisubstituted pyrimidines, piperazine substituted pyrimidine acetamides, acylaminopyrimidines, pyrimidine, pyridine, or triazine carboxamides, and mixtures or and pharmaceutically acceptable salts thereof.

In some embodiments, the permeation enhancer is selected from the group consisting of chitosan material, a fatty acid, a bile salt, a salt of fusidic acid, a polyoxyethylenesorbitan, a sodium lauryl sulfate, polyoxyethylene-9-lauryl ether (LAURETH™-9), EDTA, citric acid, a salicylate, a caprylic glyceride, a capric glyceride, sodium caprylate, sodium caprate, sodium laurate, sodium glycyrrhetinate, dipotassium glycyrrhizinate, glycyrrhetinic acid hydrogen succinate, a disodium salt, a nacylcarnitine, a cyclodextrin, a phospholipid, and mixtures thereof.

In some embodiments, the biodegradable polymer is selected from the group consisting of a polyester, a lactic acid polymer, copolymers of lactic acid and of glycolic acid, poly-ε-caprolactone (PCL), poly(anhydrides), poly(amides), poly(urethanes), poly(carbonates), poly(acetals), poly(ortho-esters), poly(glycolide-co-trimethylene carbonate), poly(dioxanone), poly(phosphoesters), poly(phosphazenes), poly(cyanoacrylate), poly(ethylene oxide), poly(propylene oxide), poly(N-isopropylacrylamide) (PNIPAAm), poly(2-(diethylamino)ethyl methacrylate) (PDEAEMA), poly(2-aminoethyl methacrylate) (PAEMA), 2 (dimethylamino)ethyl methacrylate (DMAEMA), poly(ethylene glycol) (PEG), N-(2-hydroxypropyl)methacrylamide (HPMA), poly(β-benzyl-1-aspartate) (PBLA), poly(hydroxybutyrate-co valerate), and mixtures or derivatives thereof. In some cases, the copolymers of lactic acid and of glycolic acid are selected from PLA, PGA, and PLGA. In some cases, the biodegradable polymer is PLGA, the permeation enhancer is chitosan, and the adenosine receptor antagonist is selected from the group consisting of SCH58261 and theophylline.

In another aspect, the disclosure provides an adjuvant composition comprising a particle, the particle comprising: a biodegradable polymer, a permeation enhancer, and an adenosine receptor antagonist, wherein the particle is a nanoparticle or microparticle.

In another aspect, the disclosure provides a vaccine composition comprising: a particle comprising a biodegradable polymer, n permeation enhancer, and an adenosine receptor antagonist, wherein the particle is a nanoparticle or microparticle; and an antigen.

In another aspect, the disclosure provides an oral vaccine composition comprising: a particle comprising a biodegradable polymer, a permeation enhancer, and an adenosine receptor antagonist, wherein the particle is a nanoparticle or microparticle; and an antigen.

In some embodiments of all aspects, the antigen is a disease associated protein selected from beta amyloid proteins, tau, prion proteins or its fragments, alpha-synuclein, superoxide dismutase 1, Huntingtin fragments, transthyretin, beta2-microglobulin, Apo A-1 fragments, Apo-AII, Apo AIV, TDP-43, FUS, ABri, Adan, crystallins, calcitonin, atrial natriuretic facto, prolactin, keratins, Cyrstatin C, Notch3, Glial fibrillary acidic protein (GFAP), seipin, cystic fibrosis transmembrane conductance regulator (CFTR) protein, and amylin.

In some aspects, the particle has an increased adjuvant activity.

In another aspect, the disclosure provides an adjuvant composition including a particle including a biodegradable polymer, a permeation enhancer, and an adenosine receptor antagonist and wherein the particle is a nanoparticle or microparticle.

In another aspect, the disclosure provides a vaccine composition including (i) a particle including a biodegradable polymer, a permeation enhancer, and an adenosine receptor antagonist wherein the particle is a nanoparticle or microparticle; and (ii) an antigen.

In another aspect, the disclosure provides an oral vaccine composition including (i) a particle including a biodegradable polymer, a permeation enhancer, and an adenosine receptor antagonist wherein the particle is a nanoparticle or microparticle; and (ii) an antigen.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a panel demonstrating the expression of adenosine receptors in small intestinal lamina propria dendritic cells (DC), Peyer's patches (PP), innate lymphoid cells type 3 (ILC3) and splenic CD45RB^{low} helper T (Th) cells isolated from inflamed gastric mucosa of mice.

FIGS. 2A-2D is a panel comparing the adenosine receptor subtype expression in mucosal immune/inflammatory cells. Data are expressed as the mean+/−SEM from samples prepared from multiple (>2) cell isolations.

FIG. 3 is a panel illustrating the effect of adenosine production or responsiveness on gastritis in response to infection with H. pylori. The images reflect representative views in the corpus at low and high magnification. White arrows indicate representative normal parietal cells, black arrows indicate representative loss of parietal cells or metaplasia, and gray arrows indicate representative inflammatory cells.

FIG. 4 is a graph assessing Gastritis in the corpus and antrum. Summary data are expressed as the mean+/−SEM for the inflammation assessed in all regions as calculated by Sigma Plot. N=4-8 for AR KO mice and >10 for BL/6; N=2 for UI CD73.

FIGS. 5A-5C are graphs comparing H. pylori infection burden in wild type and KO mice with and without oral immunization (CFU/g tissue) (FIG. 5A); PCR (relative units of UreE DNA)(FIG. 5B and FIG. 5C). Data are expressed as the mean+/−SEM for the inflammation assessed in all regions as calculated by Sigma Plot. N=5-34 for CFU data and 6-23 for PCR data.

FIGS. 6A-6B are graphs comparing gastritis in wildtype and KO mice. Summary data are expressed as the mean for the inflammation assessed in all regions as calculated by Sigma Plot. N=4-15 for AR KO mice and 10-17 for BL/6; N=2 for UI CD73.

FIG. 7 is a fluorescence microscopy image demonstrating the uptake of nanoparticles in macrophages. Fluorescence microscopy shows efficient uptake by macrophage cells as diffuse white intracellular particles. Nuclear areas (DNA) (light grey) were visualized by DAPI stain.

FIG. 8 is a panel of graphs demonstrating that the supplementation of the oral vaccine with a low dose of nanoparticles releasing theophylline enhances immunity by decreasing the bacterial burden. Results are the mean bacterial response in each cohort from the initial pilot experiment. High (Hi) dose=50 nM, medium (med) dose=5 nM, and low (lo) dose=0.5 nM.

FIG. 9 is a panel demonstrating that the disruption of adenosine function enhances gastritis.

FIG. 10 is an image demonstrating the sectioning of the stomach for histology and bacterial quantification.

FIG. 11 is a table demonstrating the scoring criteria used to estimate the degree of inflammation in a subject.

FIG. 12 is a table demonstrating the gastric scoring criteria.

FIGS. 13A-13B are graphs demonstrating the adjuvant effect of nanoparticles. Mice were immunized with one of three concentrations (min=1 pM, med=10 pM, max=100 pM, SCH58216) of particles, infected and assessed for bacterial burden (FIG. 13A) or gastritis (FIG. 13B).

FIG. 14 is a cartoon illustrating the nanoparticle formation setup. The high speed vortex mixing setup consists of an electric motor (Bodine Electric Co. NSE+13 LR2797) that drives a 6347 coupling unit connected to a B9045-C pump head (Tuthill, Concord, Calif.). The motor is controlled by a Staco, Inc. (Dayton, Ohio) Variable Autotransformer (Rheostat) type 3PN1010 set at 80 for 97 Volts (6000 rpm) using the 120V output. The inlet tube is more extended than the outlet tube to reach the bottom of a 50 mL conical tube. The outlet tube jets the mixing stream into the nanoparticle suspension in the tube.

### DETAILED DESCRIPTION

Provided herein is a particle including a permeation enhancer and an adenosine receptor antagonist. In some embodiments, the particle is a biodegradable particle. The particle is a nanoparticle or microparticle.

The particles described herein include an adenosine receptor antagonist. Adenosine receptor antagonists can recognize multiple adenosine receptor subtypes (i.e., adenosine A₁ receptor antagonist, adenosine A_{2A} receptor antagonist, adenosine A_{2B} receptor antagonist, or adenosine A₃ receptor antagonist), or can be selective for one or more one or more of the adenosine receptor subtypes. In some embodiments, the adenosine receptor antagonist can specifically antagonize adenosine receptor A_{2A}. In some embodiments, the antagonist is selective for adenosine receptor A_{2A}. The adenosine receptor antagonists described herein can disrupt adenosine function and/or responsiveness in a subject. Many examples of adenosine receptor antagonists are known in the art and include, for example, caffeine, theophylline, 8-phenyl theophylline, SCH58261, istradefylline, pyrazolo[4,3-e]-1,2,4-triazolo[1,5-c]pyrimidines or substituted derivatives thereof (e.g., methoxy biaryl or quinoline substitutions), SCH412348, SCH420814, fused heterocyclic pyrazolo[4,3-e]-1,2,4-triazolo[1,5-c]pyrimidines or substituted derivatives thereof (e.g., tetrahydyroisoquinoline or azaisoquinoline derivatives), aryl piperazine substituted 3H-[1,2,4]-triazolo[5,1-i]purin-5-amines, arylindenopyrimidines, arylindenopyrimidines or substituted derivatives thereof, pyrazolo[4,3-e]-1,2,4-trizolo[4,3-c]pyrimidon-3-one and thiazolotriazolopyrimidines, 1,2,4-triazolo[1,5-c]pyrimidines or substituted derivatives thereof, purinones or substituted derivatives thereof, thieno[3,2-d]pyrimidines, pyrazolo[3,4-d]pyrimidines, and 6-arylpurines, benzyl substituted triazolo[4,5-d]pyrimidines, triazolo-9H-purines, aminomethyl substituted thieno[2,3-d]pyrimidines, 2-Aminoimidazopyridines, 4-morpholino-benzothiazoles or substituted derivatives thereof, 4-Aryl and 4-morpholino substituted benzofurans, pyridone substituted pyrazines, heterocyclic substituted 2-amino-thiazoles, trisubstituted pyrimidines, piperazine substituted pyrimidine acetamides, acylaminopyrimidines, pyrimidine, pyridine, triazine carboxamides, and pharmaceutically acceptable salts thereof (Shook, et al., “Adenosine A2A Receptor Antagonists and Parkinson's Disease,” ACS Chemical Neuroscience. 2011. 2, 555-567; incorporated herein in its entirety).

In some embodiments, the particles described herein may further comprise an adenosine production enzyme (e.g., CD73) antagonist.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, Ethanedisulfonate, and 2,5-dihydroxybenzoate.

The particles described herein also include a permeation enhancer. As used herein, a “permeation enhancer” refers to a reagent that increases the permeability of mucosal cells and tissue to a therapeutic agent. For example, permeation enhancers increase the rate at which the therapeutic agent permeates through mucosal membranes and enters the bloodstream. The permeation enhancer can include, for example, various molecular weight chitosan materials, such as chitosan and N,O-carboxymethyl chitosan; poly-L-arginines; fatty acids, such as lauric acid; transkarbam; ceremides and modified ceremides; bile salts such as deoxycholate, glycolate, cholate, taurocholate, taurodeoxycholate, and glycodeoxycholate; salts of fusidic acid such as taurodihydrofusidate; polyoxyethylenesorbitan such as TWEEN™ 20 and TWEEN™ 80; sodium lauryl sulfate; polyoxyethylene-9-lauryl ether (LAURETH™-9); EDTA; citric acid; salicylates; caprylic/capric glycerides; sodium caprylate; sodium caprate; sodium laurate; sodium glycyrrhetinate; dipotassium glycyrrhizinate; glycyrrhetinic acid hydrogen succinate, disodium salt (CARBENOXOLONE™); acylcamitines such as palmitoylcamitine; cyclodextrin; and phospholipids, such as lysophosphatidylcholine. In some embodiments, the permeation enhancer includes chitosan. In some embodiments, the chitosan is acetylated. In some embodiments the permeation enhancer is a poly (acetyl or arginyl) glucosamine. In some embodiments the permeation enhancer includes mannan, glucomannan and mannose.

In some aspects, the particles described herein include a biodegradable polymer. Many suitable biodegradable polymers are known in the art, including, for example, polyesters, lactic acid polymers, copolymers of lactic acid and of glycolic acid (e.g., poly lactic acid (PLA), poly glycolic acid (PGA), or poly (lactie-co-gly colic acid) (PLGA), poly-ε-caprolactone (PCL), poly(anhydrides), poly(amides), poly(urethanes), poly(carbonates), poly(acetals), poly(ortho-esters), poly(glycolide-co-trimethylene carbonate), poly(dioxanone), poly(phosphoesters), poly(phosphazenes), poly(cyanoacrylate), poly(ethylene oxide), poly(propylene oxide), poly(N-isopropylacrylamide) (PNIPAAm), poly(2-(diethylamino)ethyl methacrylate) (PDEAEMA), poly(2-aminoethyl methacrylate) (PAEMA), 2 (dimethylamino)ethyl methacrylate (DMAEMA), poly(ethylene glycol) (PEG), N-(2-hydroxypropyl)methacrylamide (HPMA), poly(β-benzyl-1-aspartate) (PBLA), poly(hydroxybutyrate-co valerate), and mixtures or derivatives thereof. In some embodiments, the biodegradable polymer includes polyesters, lactic acid polymers, copolymers of lactic acid and of glycolic acid (e.g., PLGA), poly-ε-caprolactone (PCL), polyanhydrides, poly(amides), poly(urethanes), poly(carbonates), poly(acetals), poly(ortho-esters), and mixtures thereof. In some embodiments, the biodegradable polymer comprises poly(lactic-co-glycolic) acid, 50:50. In some embodiments, the biodegradable polymer comprises DMAPA(24)-PVAL-g-PLGA(1:7.5) or DEAPA(26)-PVAL-g-PLGA(1:10). Some embodiments also include chitosan, acetylated chitosan, or poly (acetyl, arginyl) glucosamine.

In some aspects, the particles described herein also include an antigen. Any antigen that will provoke an immune response in a human can be used in the particle compositions described herein in combination with a permeation enhancer and an adenosine receptor antagonist. By antigen, it is meant to include, but is not limited to protein, peptide, carbohydrate, glycoprotein, lipopeptide, and subunit antigens. The antigen can be derived from any source, for example, any microbial source, a bacteria (e.g., a Helicobacter pylori antigen), virus, parasite, fungus, tumor, exogenous source, endogenous source, auto-antigen source, or a neo-antigen source. In some embodiments the antigen is from a bacteria such as drug resistant bacterial strains or infectious Gram-positive and -negative strains. Bacterial antigens include, but are not limited to, H. pylori, Streptococcus pneumonia, Mycobacterium tuberculosis, Haemophilus influenza, Staphylococcus aureus, Clostridium difficile and enteric gram-negative pathogens including Escherichia, Salmonella, Shigella, Yersinia, Klebsiella, Pseudomonas, Enterobacter, Serratia, Proteus. Viral antigens include, but are not limited to, influenza viral antigens (e.g. hemagglutinin (HA) protein from influenza A, B and/or C where the influenza viral hemagglutinin protein may be at least one member selected from the group consisting of Hi, H2, H3, H5, H7 and H9, matrix 2 (M2) protein, neuraminidase), respiratory synctial virus (RSV) antigens (e.g. fusion protein, attachment glycoprotein), papillomaviral (e.g. human papilloma virus (HPV), such as an E6 protein, E7 protein, L1 protein and L2 protein), Herpes Simplex, rabies virus and flavivirus viral antigens (e.g. Dengue viral antigens, West Nile viral antigens), SARS coronavirus (SARS-CoV) antigens, human immunodeficiency virus (HIV) antigens, Flaviviridae virus (for example, Zika virus) antigens, orthomyxovirus antigens (for example, influenza virus), hepatitis viral antigens including antigens from HBV and HC. Also included are antigens of protozoan origin, for example, Plasmodium (P. vivax, P. ovale, P. malariae) antigens. Antigens used in the present compositions also include tumor antigens (i.e., an antigenic substance produced in tumor cells) and/or tumor associated antigens such as, but not limited to, AFP, CA-125, epithelial tumor antigen (ETA), tyrosinase, PSA, CEA, Mart-1, gplOO, TRP-1, MAGE, Immature laminin receptor, TAG-72, HPV E6 and E7, ING-4, Ep-CAM, EphA3, SAP-1, PRAME, SSX-2, NY-ESO-1, PAP, Mucin-1, Melanoma-associated antigen (MAGE), Brother of regulator of imprinted sites (BORIS), and PSMA. Antigens used in the present compositions also include disease associated proteins such as, for example, beta amyloid proteins, tau, prion proteins or its fragments, α-synuclein, superoxide dismutase 1, Huntingtin fragments, transthyretin, β-microglobulin, Apo A-1 fragments, Apo-AII, Apo AIV, TDP-43, FUS, ABri, Adan, crystallins, calcitonin, atrial natriuretic facto, prolactin, keratins, Cyrstatin C, Notch3, Glial fibrillary acidic protein (GFAP), seipin, cystic fibrosis transmembrane conductance regulator (CFTR) protein, and amylin. These antigens can further include modifications, deletions, additions and substitutions to the native antigen molecule. In some cases the particle may include a H. pylori antigen. In some embodiments, the antigen is incorporated within and throughout the particle (i.e., absorbed throughout the particle). In some embodiments, the antigen is disposed (i.e., presented, attached, loaded) on the surface of the particle. Also included are the antigenic compositions themselves. In some embodiments, the antigenic compositions comprise the antigens described herein.

In some aspects, the compositions can additionally include a therapeutic agent. For example, the compositions described herein can include an antimicrobial agent (e.g., an, antibiotic agent, an anti-fungal agent, or an anti-viral agent, an anti-cancer agent, an anti-tumor agent, signaling proteins, ligands to target specific cells, small molecules, nucleic acids, antibodies or fragments thereof. For example the anti-cancer agents described herein can include Colchicine, Vincristine, Vinblastine, anti-CD47 antibodies, TLR4 agonists (e.g., HMGB1, HMGB1 peptides, SAFFLFCSE (UC1018)), Hp91, small molecule TGF-beta inhibitors, (e.g., SB431542, GW788388), L-1MT, antibodies to TGF-beta (e.g., 1D11) and TLR7 ligands (e.g., Imiquimod). In some embodiments, the particles include a therapeutic agent incorporated within and throughout the particle. In some embodiments, the therapeutic agent is presented disposed (i.e., presented) on the surface of the particle.

Further, in some aspects, the particles described herein can include a cell targeting (binding) moiety. This moiety can be specific for a particular cell type, receptor, or other target moiety in the subject or can include a specific cellular import signal or sequence. For example, the targeting moiety can be a cell-targeting moiety including, for example, a phagocytic cell-targeting moiety, a macrophage-targeting moiety, a tumor cell-targeting moiety, an epithelial cell (including “M” cell) targeting moiety, and a dendritic cell-targeting moiety, a myeloid cell moiety, a Natural killer cell moiety, a T-cell moiety, a glial cell moiety and a B-cell moiety. Suitable targeting (binding) moieties for selective targeting can be developed, or are available or known. For example, a targeting moiety can be an antibody, antibody fragment, bispecific or other multivalent antibody, or other antibody-based molecule or compound. The antibody can be of various isotypes, preferably IgG1, IgG2a, IgG3, IgG4, and IgA, and can be a chimeric human-mouse, a chimeric human-primate, a humanized (human framework and murine hypervariable (CDR) regions), or fully human MAbs, as well as variations thereof. Other binding moieties known in the art, such as aptamers, avimers or targeting peptides, may be used. Diseases or conditions against which such targeting moieties exist are, for example, cancer, immune dysregulatory conditions, including autoimmune diseases and inflammatory diseases, and diseases caused by infectious organisms.

In some embodiments, the particle further includes a dye. For example, the particle can further include a lipophilic tracer dye such as DiD dye (1,1″-dioctadecyl-3,3, 3″,3″-tetramethylindodicarbocyanine), DiO dye (3,3′-dioctadecyloxacarbocyanine), DiA dye (4-(4-(dihexadecylamino)styryl)-N-methylpyridinium), DiI dye ((2Z)-2-[(E)-3-(3,3-dimethyl-1-octadecylindol-1-ium-2-yl)prop-2-enylidene]-3,3-dimethyl-1-octadecylindole; perchlorate; CAS No. 41085-99-8), and DiR dye (1,1′-dioctadecyl-3,3,3′,3′-tetramethylindotricarbocyanine), which are commercially available from Life Technologies®. The dyes described herein can have various emission wavelengths. One of skill in the art would understand that the dyes described herein have various purposes including but not limited to particle identification, size determination, tracking, and quantification in vitro and in vivo.

As used herein, the term “nanoparticle” refers to a particle having an average diameter of about 0.5 nm to about 1 m. In some embodiments, the nanoparticle has an average diameter of about 5 nm to about 950 nm, about 50 nm to about 900 nm, about 100 nm to about 800 nm, about 150 nm to about 750 nm, about 200 nm to about 700 nm, about 300 nm to about 600 nm, or about 400 nm to about 500 nm.

As used herein, the term “microparticle” refers to a particle having an average diameter of about 1 μm to about 1 mm in diameter. In some embodiments, the microparticle has an average diameter of from about 1 μm to about 1,000 μm, about 5 μm to about 950, about 50 μm to about 900, about 100 μm to about 800, about 200 μm to about 700, about 300 μm to about 600, or about 400 μm to about 500.

In some embodiments, the particle has an average diameter of about 10 nm to about 80 μm, about 200 nm to about 580 μm. For example, the particle can have an average diameter of about 10 nm to about 1,000 nm. In some embodiments, the particle has an average diameter of about 1 μm to about 80 μm.

Additionally, provided herein is a particle including PLGA, a permeation enhancer including chitosan, and an adenosine receptor antagonist comprising SCH58261 and wherein the particle is a nanoparticle or microparticle. Additionally, provided herein is a particle including PLGA, a permeation enhancer including chitosan, and an adenosine receptor antagonist comprising theophylline and wherein the particle is a nanoparticle or microparticle.

Also included are the pharmaceutical compositions comprising the particles themselves. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions and adjuvant compositions described herein comprising the particles are formulated to be compatible with its intended route of administration. Examples of routes of administration include parenteral, e.g., intravenous, intradermal, subcutaneous, oral (e.g., inhalation), transdermal (topical), transmucosal, intratumoral and rectal administration. In some embodiments, the pharmaceutical compositions comprising the particles described herein are formulated for oral administration. In some embodiments, the pharmaceutical compositions comprising the particles described herein are formulated for intratumoral administration. In some embodiments, the adjuvant or pharmaceutical compositions described herein is presented disposed (i.e., delivered) into a tumor. In some embodiments in the adjuvant can be delivered p.o, i.p. s.c. or i.v. sublingual, lung inhalation, nasal administration, suppositories, eye drops or other means of administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions of the particles suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Formulations of the particles described herein can be prepared to enable freeze-drying. These formulations can include a buffer, a cryoprotective agent, a lyoprotective agent, a bulking matrix, a caking agent and/or an emulsifying agent. The lyoprotectants described herein can include disaccharides, for example, sucrose and trehalose. The lyoprotectants described herein can also include glycerol. Matrix forming additives or excipients described herein can include mannitol and proteins such as serum albumin.

Sterile injectable solutions of the particles described herein can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions of the particles can include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the particles can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a pharmaceutical compositions comprising the particles as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic particles are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Also included are the adjuvant compositions comprising the particles described herein. “Adjuvant” refers to any substance that assists or modifies the immunological action of a pharmaceutical compositions, including but not limited to agents that increase or diversify the immune response to an antigen or agents that increase the efficacy of a vaccine. In some embodiments, the adjuvant composition further comprises immunostimulating agents, including, for example: an aluminum salt, complete Freund's adjuvant (CFA), incomplete Freund's adjuvant (IFA), muramyl dipeptide (MDP), MF59, QS21, bacterial toxins or toxoids known to enhance immunity, biological response modifiers, or immunostimulating complexes known in the art.

Additionally, provided herein is an adjuvant composition including a particle including a biodegradable polymer, a permeation enhancer, and an adenosine receptor antagonist and wherein the particle is a nanoparticle or microparticle.

In some embodiments, the particle has an increased adjuvant activity as compared to a similar particle without the adenosine receptor antagonist.

Also included are vaccine compositions that comprise the particles described herein. These vaccines comprise the particles described herein and an antigen. The vaccine composition can be formulated for any route of administration described herein, including parenteral, e.g., intravenous, intradermal, subcutaneous, oral (e.g., inhalation), transdermal (topical), transmucosal, rectal and intratumoral administration.

Additionally, provided herein is a vaccine composition including (i) a particle including a biodegradable polymer, a permeation enhancer, and an adenosine receptor antagonist wherein the particle is a nanoparticle or microparticle; and (ii) an antigen.

Additionally, provided herein is an oral vaccine composition including (i) a particle including a biodegradable polymer, a permeation enhancer, and an adenosine receptor antagonist wherein the particle is a nanoparticle or microparticle; and (ii) an antigen.

Additionally, provided herein is a method of therapeutically or prophylactically treating an individual in need thereof comprising administering an effective amount of a particle including a biodegradable polymer, a permeation enhancer, and an adenosine receptor antagonist, wherein the particle is a nanoparticle or microparticle, to elicit an immune response.

Additionally, provided herein is a method of treating a H. pylori infection in a mammalian subject comprising administering a particle including a biodegradable polymer, a permeation enhancer, and an adenosine receptor antagonist, wherein the particle is a nanoparticle or microparticle and wherein the particle further includes an a Helicobacter pylori antigen.

Also included are methods for the treatment of diseases and disorders associated with the anti-inflammatory effects of the regulatory T cell-derived mediator adenosine. The disease or disorder can be, for example, an infectious disease, e.g. persistent infection (e.g., bacterial, fungal, viral, or parasitic) or cancer (e.g. a tumor, or non-cancerous tumor, carcinoma). Generally, the methods include administering a therapeutically effective amount of the particles as described herein, to a subject who is in need of, or who has been determined to be in need of, such treatment. In some embodiments, the subject in need thereof has been diagnosed. In some embodiments, the subject is treated prophylactically or before diagnosis.

As used in this context, to “treat” means to ameliorate at least one symptom of the disease or disorder. Often, treatment results in blocking of the anti-inflammatory effects of adenosine; thus, a treatment can result in enhanced immunity and/or clearance of an infection.

The compositions described herein can be formulated for any form of administration, and can be administered by any method suitable for administration of the pharmaceutical composition, vaccine compositions, or adjuvant compositions described herein.

The compositions described herein can be administered before, after, or concurrently with other treatments. The compositions can also be administered prophylactically to a subject in need thereof.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

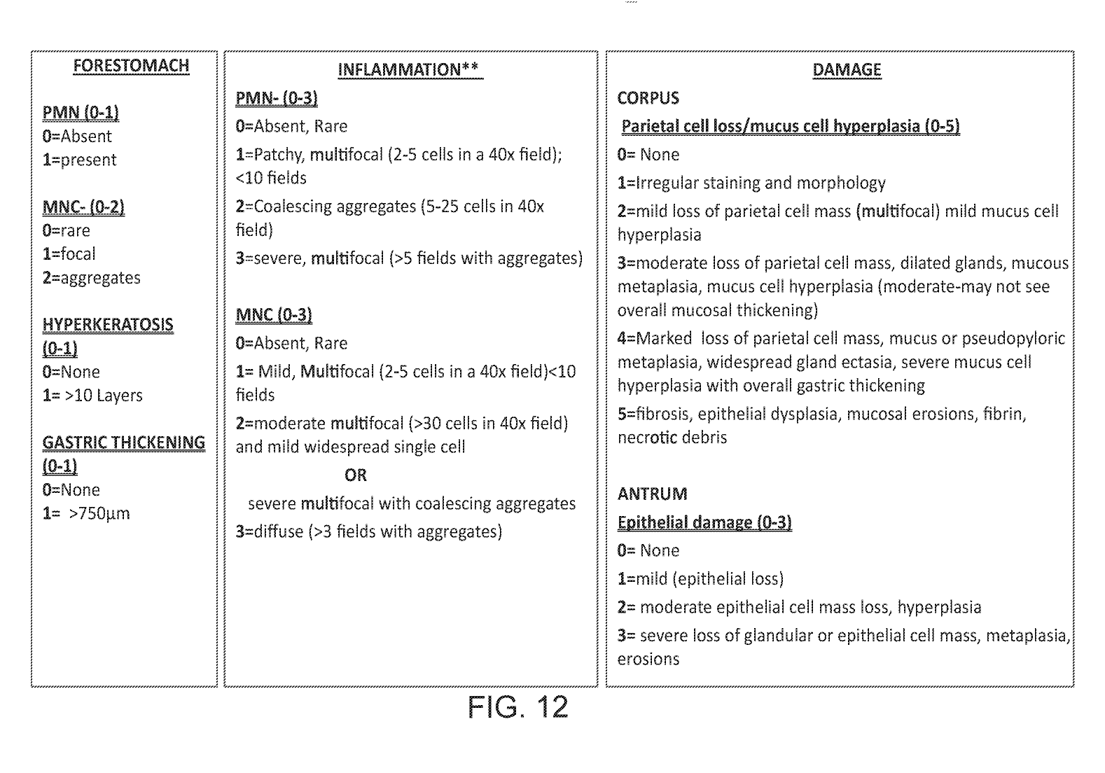

As used herein, the term “gastritis score” refers to a histopathological score to quantify a degree of gastritis. The table in FIG. 12 provides an exemplary method for determine the gastritis score (i.e., the degree of gastritis) for a subject.

Also included are methods of treating a subject. In some embodiments the methods of treating a subject comprise administering the particles or pharmaceutical compositions comprising the particles described herein. In some embodiments the methods of treating a subject comprise administering an adjuvant composition described herein. In some embodiments the methods of treating a subject comprise co-administering an antigenic composition described herein with an adjuvant composition described herein. In some embodiments, the method of treating a subject comprises administering a vaccine composition described herein. In some embodiments, the method of treating a subject comprises co-administering an antigenic composition described herein with a vaccine composition described herein. In some embodiments the methods of treating a subject comprise co-administering an adjuvant composition described herein with the vaccine composition described herein. In some embodiments, the adjuvant composition described herein is administered (i.e., delivered, presented disposed) into a tumor.

Also included are methods of enhancing an immune response in a subject. In some embodiments, the method of enhancing an immune response comprise administering to the subject a therapeutically effective amount of the particles described herein or a pharmaceutical compositions comprising a therapeutically effective amount of the particles described herein. In some embodiments the method of enhancing an immune response comprise administering an adjuvant composition described herein. In some embodiments the method of enhancing an immune response comprise co-administering an antigenic composition described herein with an adjuvant composition described herein. In some embodiments, the method of enhancing an immune response comprises administering a vaccine composition described herein. In some embodiments, the method of enhancing an immune response comprises co-administering an antigenic composition described herein with a vaccine composition described herein. In some embodiments the methods of enhancing an immune response comprise co-administering an adjuvant composition described herein with the vaccine composition described herein.

In some embodiments, the particles block adenosine receptor function in a subject and the blocking is sufficient to enhance an immune response in the subject. In some embodiments, the particles block adenosine activity in the cell and the blocking is sufficient to enhance an immune response in the subject. In some embodiments, the particles increase APC activation. In some embodiments, the particles increase CTL activation.

Also included are methods of increasing the efficacy of a vaccine. In some embodiments the method of increasing the efficacy of a vaccine comprises administering the adjuvant composition described herein. In some embodiments the method of increasing the efficacy of a vaccine comprises co-administering the adjuvant composition described herein with an antigenic composition or antigen described herein.

Also included are methods of treating a tumor in a subject in need thereof. In some embodiments the methods of treating a tumor comprise administering to the subject a therapeutically effective amount of the particles described herein or a pharmaceutical composition comprising a therapeutically effective amount of the particles described herein. In some embodiments the method of treating a tumor comprise administering an adjuvant composition described herein. In some embodiments the method of treating a tumor comprise co-administering an antigenic composition described herein with an adjuvant composition described herein. In some embodiments, the method of treating a tumor comprises administering a vaccine composition described herein. In some embodiments, the method of treating a tumor comprises co-administering an antigenic composition described herein with a vaccine composition described herein. In some embodiments the methods of treating a tumor comprise co-administering an adjuvant composition described herein with the vaccine composition described herein. In some embodiments, the method of treating a tumor further comprises co-administering an immunotherapy, including, for example: immune modulators (e.g., immune inhibitors and immune enhancers). In some embodiments, the method of treating a tumor further comprises co-administering an anti-cancer or anti-tumor agent.

Also included are methods of vaccinating a subject. In some embodiments the methods of vaccinating a subject comprise co-administering to the subject a therapeutically effective amount of the particles described herein or a pharmaceutical compositions comprising a therapeutically effective amount of the particles described herein with the antigenic compositions described here. The antigenic compositions can be administered before, after, or concurrently with the administering of the particle or pharmaceutical compostions. In some embodiments the methods of vaccinating a subject comprise administering particles comprising antigens as described herein. In some embodiments the methods of vaccinating a subject comprise administering the pharmaceutical compositions described herein. In some embodiments the methods of vaccinating a subject comprise administering the adjuvant compositions described herein. In some embodiments the methods of vaccinating a subject comprise administering the vaccine compositions described herein.

### EXAMPLES

The invention is further described in the following examples, which do not limit the scope of the invention described in the claims.

### Materials

PLGA (50:50 Poly(DL-lactide-co-glycolide), ester terminated, with an inherent viscosity range of 0.95-1.20 dL/gm in HIFP was purchased from Durect Corp (Product No. B6010-4P), Pelham Ala.

Chitosan (β-(1-4)-linked D-glucosamine and N-acetyl-D-glucosamine; low molecular weight, Brookfield Viscosity 20,000 cps) was purchased from Sigma-Aldrich (Product No. 448869).

Acetic Acid, Glacial, was purchased from Fisher Scientific (Product No. A35-500).

Ethyl Acetate (CAS141-78-6), was purchased from Fisher Scientific (Cat. No. E196-4).

DMSO (dimethyl sulfoxide) (CAS 67-67-5), was purchased from Fisher Scientific (Product No. BP231-1).

PVA (Poly(vinyl alcohol) (CAS 9002-89-5), 87-89% hydrolyzed, was purchased from Sigma-Aldrich (Cat. No. 363170-500 g).

SCH58261 (5-Amino-7-(2-phenylethyl)-2-(2-furyl)-pyrazolo(4,3-e)-1,2,4-triazolo(1,5-c)pyrimidine) was purchased from Tocris (Cat. No. 2270).

Theophylline (1,3-Dimethyl-7H-purine-2,6-dione), Anhydrous (CAS 58-55-9) was purchased from Spectrum Chemicals (Product No. TH110).

DiD (DiIC18(5) solid (1,1″-dioctadecyl-3,3, 3″,3″-tetramethylindodicarbocyanine, 4-chlorobenzenesulfonate salt) was purchased from Invitrogen (Thermo Fisher)(Product No. D7757).

SWFI (Sterile Water for Inj., USP) was from Hospira, Inc., Forest Lake, Ill.

### Methods

SCH58261 Nanoparticles with Chitosan

ORGANIC Phase: An ORGANIC phase solution was prepared by dissolving PLGA (50:50) in the amount of 200 mg in 10 mL ethyl acetate. To this solution, 0.05 mL of a 5 mg/mL SCH58261 solution in DMSO and 0.1 mL of a 0.4 mg solution of DiD lipophilic tracer dye in DMSO was added.

AQUEOUS Phase: An AQUEOUS phase solution was prepared by adding 15 mg of chitosan and 15 microliters of acetic acid was added to 9 mL SWFI. After dissolving the chitosan, 1 mL of 1% PVA in Sterile Water for Injection, USP (SWFI, or equivalent) was added.

The particle charge can be modulated by altering the chitosan content. For example, 30 mg of chitosan and 30 microliters of acetic acid were added to 9 ml SWFI and the charge (the zeta potential) of the particle was increased.

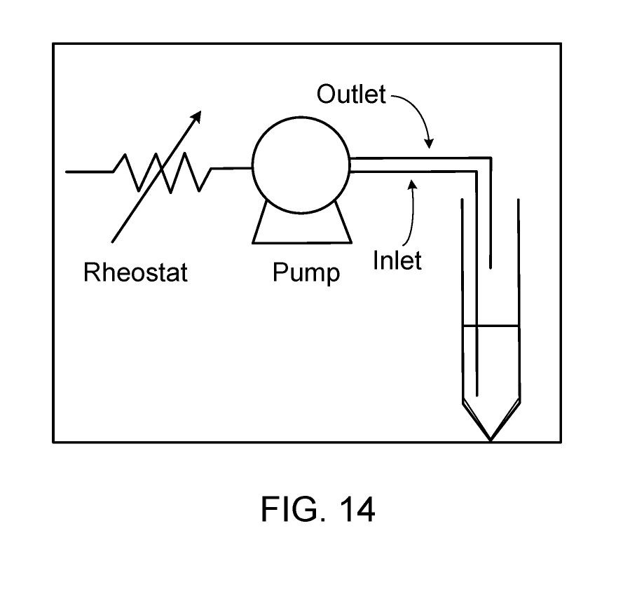

Nanoparticle Formation: The ORGANIC Phase is poured into the AQUEOUS Phase and vigorously mixed by shaking. The suspension was then submitted to high speed vortex mixing for 3 minutes, to which 20 mL of 0.1% PVA in SWFI was added while continuing to vortex (FIG. 14). The suspension was transferred to a beaker on a magnetic stirrer and an additional 60 mL of 0.1% PVA, SWFI was added. The suspension was stirred overnight to evaporate the ethyl acetate. Aggregates were pelleted by low speed centrifugation (RCF=70) and the supernatant centrifuged at 230×g for 10 min. The pellet was taken up in 10 mL of SWFI, 0.1% PVA and pelleted by centrifugation, then taken up in 2 mL of SWFI, 0.1% PVA aliquoted and stored at −20° C. The nanoparticles remaining in suspension were pelleted at 1380×g for 10 min. The size of the collected particles is dependent on the RCF (relative centrifugal force) and time of centrifugation. Any residual aggregates are removed by centrifugation at low speed (e.g., 70×g) for 1 min.

Dilution of Nanoparticles: The nanoparticle suspension was serially diluted with SWFI, 0.1% PVA to 2× the desired final concentration. An equal volume of 2× concentrated buffer (e.g. 2×PBS) is added to make the final concentration. To minimize aggregates, the nanoparticle pellets can be resuspended and diluted into 50 mM citrate pH 3.3, 0.1% PVA. In some instances, the final dilution was performed with 0.9% saline, 5 mM phosphate buffer, pH 6.5. In some cases the pH of the solution should not exceed 6.5.

### Theophylline Nanoparticles

ORGANIC Phase: An ORGANIC phase solution was prepared by dissolving PLGA (50:50) in the amount of 200 mg in 9.5 mL ethyl acetate, after which 0.5 mL of 30 mg/mL theophylline dissolved in DMSO was added. To this, 0.1 mL of a 0.4 mg solution of DiD in DMSO was added.

AQUEOUS Phase: An AQUEOUS phase solution was prepared by adding 30 mg of chitosan and 30 microliters of glacial acetic acid to 9 mL SWFI. After dissolving the chitosan, 1 mL of 1% PVA and 80 mg of theophylline was added to a final concentration of 8 mg/mL theophylline.

Nanoparticle Formation: The ORGANIC Phase is poured into the AQUEOUS Phase and vigorously mixed. The suspension was then submitted to high speed vortex mixing for 3 minutes, to which 20 mL of 0.1% PVA and 8 mg/mL theophylline in SWFI was added while continuing mixing (FIG. 14). The suspension was transferred to a beaker on a magnetic stirrer and an additional 60 mL of 0.1% PVA, SWFI, 8 mg/mL theophylline was added. The suspension was stirred overnight to evaporate the ethyl acetate. The nanoparticles were pelleted by centrifugation and washed with 4×10 mL volumes of 0.1% PVA, SWFI. Injection. The final wash pellet was taken up in 2 mL of SWFI, 0.1% PVA, aliquoted and stored at −20° C. The nanoparticle suspension was serially diluted with SWFI, 0.1% PVA to 2× the desired final concentration. An equal volume of 2× concentrated buffer (e.g., 2×PBS) is added to make the final concentration.

Note: For control nanoparticles no SCH58261 or Theophylline was added. EA: ethyl acetate solution, pre-filtered; AQ: aqueous solution; SWFI: Sterile Water for Injection, USP; PLGA: poly lactic-co-glycolic acid, 50:50.

Theophylline Nanoparticles with Chitosan

ORGANIC Phase: An ORGANIC phase solution was prepared by dissolving PLGA (50:50) in the amount of 200 mg in 9.5 mL ethyl acetate, after which 0.5 mL of 30 mg/mL theophylline dissolved in DMSO was added. To this, 0.1 mL of a 0.4 mg solution of DiD in DMSO was added.

AQUEOUS Phase: An AQUEOUS phase solution was prepared by adding 28.5 mg of chitosan and 50 mg of Theophylline and 15 microliters of glacial acetic acid was added to 8.5 mL SWFI. After the chitosan and theophylline dissolve, 1 mL of 1% PVA was added.

Nanoparticle Formation: The ORGANIC Phase is poured into the AQUEOUS Phase and vigorously mixed. The suspension was then submitted to high speed vortex mixing for 3 minutes and then transferred to a beaker with stirring bar in a 40° C. water bath (FIG. 14). A solution of 30 mL of distilled water, 0.1% PVA containing 8 mg/mL theophylline was added to the beaker while continuing mixing for 1 hour at 40° C. The suspension was stirred overnight at room temperature. The nanoparticles were pelleted by centrifugation at 4000 rpm (RCF=230×g) 5 min. The pellets were resuspended in 2.7 mL of distilled water and refrigerated for 1 hr. and centrifuged at 4000 rpm (230×g) for 5 min. The supernatant was removed and the pellet was resuspended in 13 mL distilled water, 0.1% PVA and centrifuged at 4000 rpm (230× g, 5 min). The pellet was resuspended in 2.2 mL SWFI, 0.1% PVA, aliquoted (100 μL/vial) dried under vacuum (approx. 5 mg/vial dry weight). [Alternatively, instead of drying, the aliquots may be kept frozen (e.g., −20° C.).] The theophylline content was 1.18 μg/mg of PLGA polymer. The nanoparticles are reconstituted in SWFI, 0.1% PVA. The nanoparticle suspension is serially diluted with SWFI, 0.1% PVA to 2× the desired final concentration. An equal volume of 2× concentrated buffer (e.g., 2×PBS) is added to make the final concentration.

Note: For control nanoparticles, no SCH58261 or Theophylline was added.

### Characterization of Nanoparticles:

SCH58261 or Theophylline content: The nanoparticle suspension (drug or control nanoparticles, 200 microliter aliquots) was dried under vacuum, weighed and dissolved in 1 mL of DMSO. SCH58261 or Theophylline content (μg/mg polymer) was determined on a UV/Vis spectrophotometer (Ultrospec 2100pro UV Spectrophotometer (GE Healthcare) with Swift II software) using a quartz cuvette of 1 cm path length. Measurements were blanked against weight-matched control nanoparticles or a solution of 5 mg/mL PLGA in DMSO. Theophylline was measured by determining the absorbance at λmax A₂₇₄, ε=0.0527. SCH58261 was measured by determining the absorbance at λmax A₂₈₅, ε=0.061. The concentration in micrograms per mL is determined by dividing the absorbance measurement by c, the extinction coefficient.

Size and Zeta Potential: The average particle sizes and zeta potential were analyzed by Composix, San Diego using a Malvern DLS/Zeta Sizer.

Reference: Kumar MNV, Bakowsky U, Lehr C M. (2004) Preparation and characterization of cationic PLGA nanospheres as DNA carriers. Biomaterials 2004; 25: 1771-1777; incorporated herein in its entirety.

### Bacterial Count by Plating

Stomach tissue in Brucella Broth and weight of tissue was recorded. Tissue was then homogenized and serially diluted in PBS. Dilution is plated on the H. pylori-selective Blood Agar Plates (Pappo J. et al. Infection and Immunity 1999; 67(1): 337-341) (Tryptic soy agar plates containing: 5% Sheep blood, vancomycin, polymyxin B, bacitracin, nalidixic acid and amphotericin B).

Plates were then incubated 5-8 days in a 10% CO₂ incubator. Colonies were counted for each dilution and CFU (colony forming unit)/g of tissue was calculated.

### Bacterial Count by PCR

Quantification of the UreB (Urease subunit beta) gene was used estimate the number of H. pylori bacteria. DNA was isolated from the stomach tissue. UreB specific primers were used to detect the level of H. pylori in the stomach tissue by quantitative PCR using SYBR Green. Count value was then noted for UreB and the internal GAPDH control.

### Histological Samples

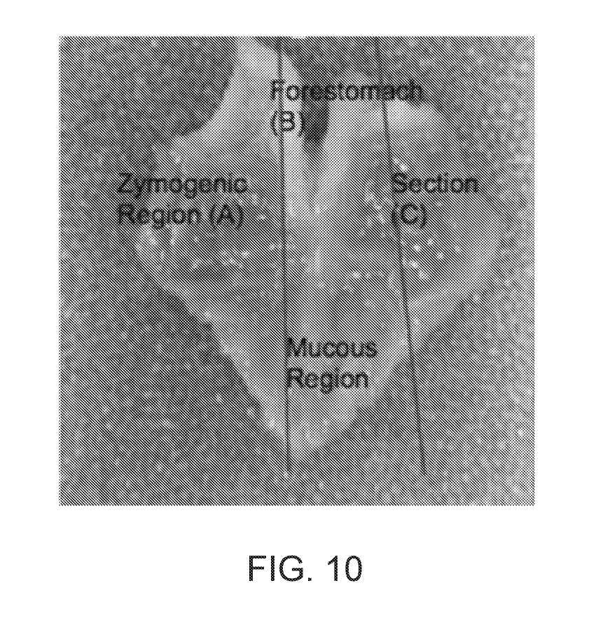

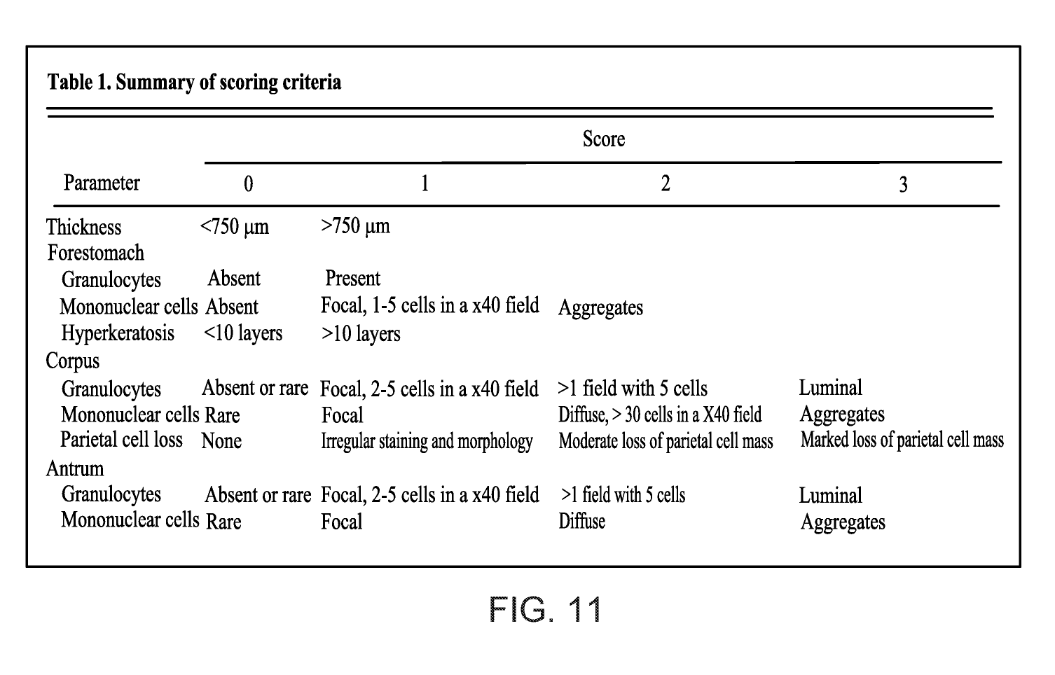

Mice were vaccinated once a week for 4 weeks, followed by 3 infections of H. pylori given every other day over the course of a week via oral gavage. At 6 weeks post-infection, mice were euthanized and the stomach, spleen, and lymph nodes were harvested. One section of the stomach was used for histology (FIG. 11 and FIG. 12) and other areas were used for quantifying bacteria (FIG. 10). Bacteria were quantified by culture and PCR. Plating provided an estimate of viable bacteria.

### Preparation of Vaccine

H. pylori were solubilized by sonication. The soluble H. pylori (100 μg) was then administered with 5 μg cholera toxin concomitantly with the nanoparticles by gavage. Immunization was repeated weekly.

### Protocol for Immunization

Mice were orally immunized (H. pylori sonicate plus cholera toxin with or without the nanoparticles) two times, rested for 1 week and then challenged by gavage three times (every other day) with 1×10⁸ CFU H. pylori (SS1=I) or left uninfected (UI) and housed for another 6 weeks. At that time, mice were euthanized and tissues were collected

### Example 1: Determining the Phenotype of T Helper Cells in H. pylori Uninfected and Infected Mice

Regulatory T cells (Tregs) contribute to persistent infection with H. pylori. Gastric Treg express the A2A adenosine receptor (A_{2A}AR) (Alam M S, Kurtz C C, Wilson J M, Burnette B R, Wiznerowicz E B, Ross W G, et al. A2A adenosine receptor (AR) activation inhibits pro-inflammatory cytokine production by human CD4+ helper T cells and regulates Helicobacter-induced gastritis and bacterial persistence. Mucosal Immunol. 2009; 2(3):232-42; incorporated herein in its entirety) that regulates their induction and the optimal expression of Foxp3 (Zarek P E, Huang C T, Lutz E R, Kowalski J, Horton M R, Linden J, et al. A2A receptor signaling promotes peripheral tolerance by inducing T cell anergy and the generation of adaptive regulatory T cells. Blood. 2008; 111:251-9: Available from: PM:17909080; incorporated herein in its entirety). Wildtype, A_{2A}AR, A_{2B}AR, A_{2A}/_{2B}AR double knock out (DKO) or CD73 KO mice of different ages (neonatal, 7, 21, 42 or 8-12 weeks of age) were infected with H. pylori. The phenotype of T helper cells (Th cells) in the spleen and gastric lymph nodes were assessed in uninfected and infected mice with attention being paid to Treg. The gastric lymph nodes were used as the source of cells as the inventors have previously shown that the composition of this population is almost identical to the cells isolated from the gastric mucosa (Ernst P B, Erickson L D, Loo W M, Scott K G, Wiznerowicz E B, Brown C C, et al. Spontaneous autoimmune gastritis and hypochlorhydria are manifest in the ileitis-prone SAMP1/YitFcs mice. Am J Physiol Gastrointest Liver Physiol. 2012; 302(1):G105-15; incorporated herein in its entirety). We used markers to define the lineage (CD3, CD4) and major Th cell subsets including Th1 (Tbet, IFN-γ), Th17 (Rorγt, IL-17A) and Treg (FoxP3). Although the degree of inflammation changed markedly among strains of mice in response to infection at different ages, there was no appreciable difference in the proportion of T helper cell (“Th cell”) subsets. A mixed infiltrate of Th1, Th17 and Treg of comparable proportions were found in all cohorts with only the absolute numbers of cells changing as reflected in the gastritis.

### Example 2: Determining the Role of Adenosine Receptor Subtypes in Adenosine-Induced Persistence

Immune/inflammatory cells contributing to the clearance of H. pylori differ in their expression of adenosine receptor subtypes. In developing an adjuvant strategy, one can choose between nonspecific or selective adenosine antagonists by identifying the effect different adenosine receptors have on gastritis and by determining the receptors expressed by the immune cells responsible for protection. In developing an adjuvant strategy, one can choose from nonspecific or selective adenosine antagonists by identifying the optimal cellular target.

T cells and B cells were isolated from inflamed gastric mucosa of mice while PMN and monocytes (myeloid cells) were prepared from systemic sites and resting or activated cells were assayed for the expression of A₁, A_{2A}, A_{2B} or A₃ adenosine receptors by real-time RT PCR. These studies identified the receptor subtypes expressed on the majority of the effector cells tested and this information can guide the choice of adenosine receptor antagonist (e.g., theophylline or ZM241395) or KO mice. For example, if expression is limited to A_{2A}, A_{2B} then ZM241395 can be tested as the antagonist, while the presence of A1 and/or A3 receptors can require theophylline to antagonize as it can target all 4 receptor subtypes.

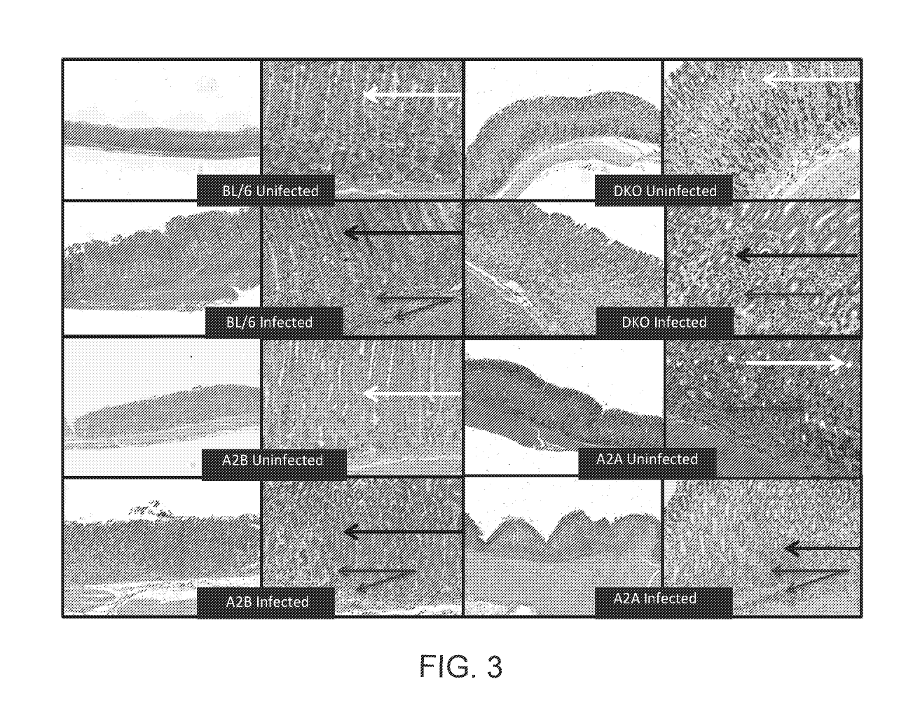

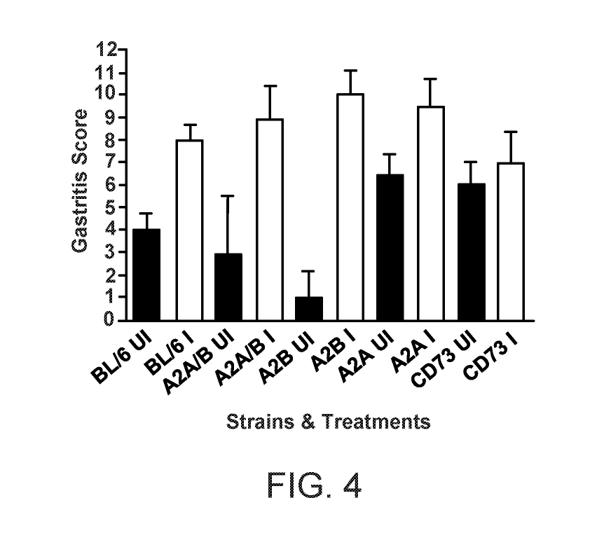

To address which adenosine receptor subtype should be inhibited with the drugs provided by the nanoparticles, we initially assessed gastritis in uninfected and infected mice that have normal (C57BL/6 wildtype) or comprised responses to (A_{2A}/A_{2B} DKO, A_{2A}KO and A_{2B}KO) or production of (CD73KO) adenosine. As shown in FIG. 3 and FIG. 4, gastritis was increased in all strains after infection. FIG. 3 shows photomicrographs of tissue sections from the corpus region of the stomach after staining with hematoxylin and eosin. Images were captured at two different magnifications (1.2× and 15×), white arrows indicate representative normal parietal cells, black arrows indicate representative loss of parietal cells or metaplasia, and gray arrows indicate representative inflammatory cells. All infected strains had an increase in inflammatory cells throughout the mucosa, often with associated damage to parietal cells compared to the uninfected samples. Data from multiple samples are summarized in FIG. 4, confirming that gastritis was increased in all strains after infection.

The constitutive gastritis was modest in uninfected wildtype BL/6 mice and in the mice lacking the A_{2B}AR including the A_{2A}/A_{2B} DKO and A_{2B}KO mice. Gastritis in uninfected mice was most apparent in mice lacking the ability to produce adenosine (CD73KO) and respond through the A_{2A}AR (A_{2A}AR KO). These data suggest that the A_{2A}AR has more of an effect on the control of gastritis before infection and thus, may be a preferred target when trying to enhance host responses by mucosal immunization.

To further implicate a specific adenosine receptor subtype, adenosine receptor expression on cells responsible for immunity to H. pylori was assessed. The prioritization that guided the order in which the different lineages were studied was based on the published evidence supporting their role in immunity to H. pylori. Thus, helper Th cells that are necessary for vaccine-based protection were studied first (Ermak T H, Giannasca P J, Nichols R, Myers G A, Nedrud J, Weltzin R, et al. Immunization of mice with urease vaccine affords protection against Helicobacter pylori infection in the absence of antibodies and is mediated by MHC class II-restricted responses. Journal of Experimental Medicine. 1998; 188:2277-88; incorporated herein in its entirety).

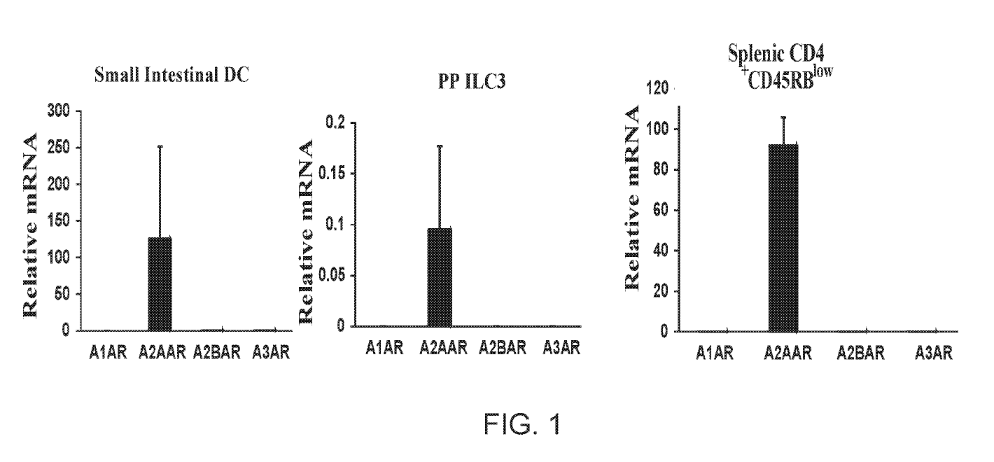

Since innate lymphoid cells (ILC) resemble Th cells and have a role in resistance to other bacteria (Spits H, Artis D, Colonna M, Diefenbach A, Di Santo J P, Eberl G, et al. Innate lymphoid cells—a proposal for uniform nomenclature. Nat Rev Immunol. 2013; 13(2): 145-9; incorporated herein in its entirety), ILC cells were isolated from mucosal tissues and evaluated for their contribution to gastrointestinal immunity. Like Th cell subsets, ILC subtypes (ILC1, ILC2 and ILC3) are described based on the expression of surface markers, transcription factors and cytokines. ILC1 are typically found in systemic tissues such as the spleen while ILC3 are a major subset in the gastrointestinal tract. The ILC represent less than 1% of all white cells in the mucosa so this is a challenging task (Drygiannakis I, Kurtz, C. C., Klann, J., Farrow, N. E., Thai, R., Wilson, J. M., Borowitz, M., Kediaris, V., Ware, C. F., Ernst, P. B. CD73 controls the fate of intestinal Th cells and ILC3 during Th cell-mediated colitis. In Preparation. 2014, and Kurtz C C, Drygiannakis, I. Naganuma, M., Feldman, S., Bakiaris, V., Linden, J., Ware, C. F., Ernst, P. B. Extracellular adenosine regulates colitis through effects on lymphoid and non-lymphoid cells. Amer J Physiol Gastrointest Liver Physiol 2014; 307:G338-G46; incorporated herein in their entirety). Small intestinal lamina propria dendritic cells (DC), Peyer's patches (PP) innate lymphoid cells type 3 (ILC3) and splenic CD45RB^{low} helper T (Th) cells were sorted by flow cytometry (DC: live CD11b⁺CD11c^{high}; ILC3: CD11c⁻I-Ab⁻CD49b⁻BTLA⁻CD11b⁻TcRβ⁻Thy1.2⁺CD127⁺ or I-Ab⁻CD49b⁻BTLA⁻CD11b⁻TcRβ⁻Thy1.2⁺CD127⁺Rorγ(t)⁺ from Rorgt-GFP mice; Treg: CD4⁺CD45RB^{low}). Adenosine receptor mRNA expression was assayed by reverse transcription quantitative PCR. A_{2A} adenosine receptor (A_{2A}AR) was the most abundant of the four adenosine receptors (FIG. 1).

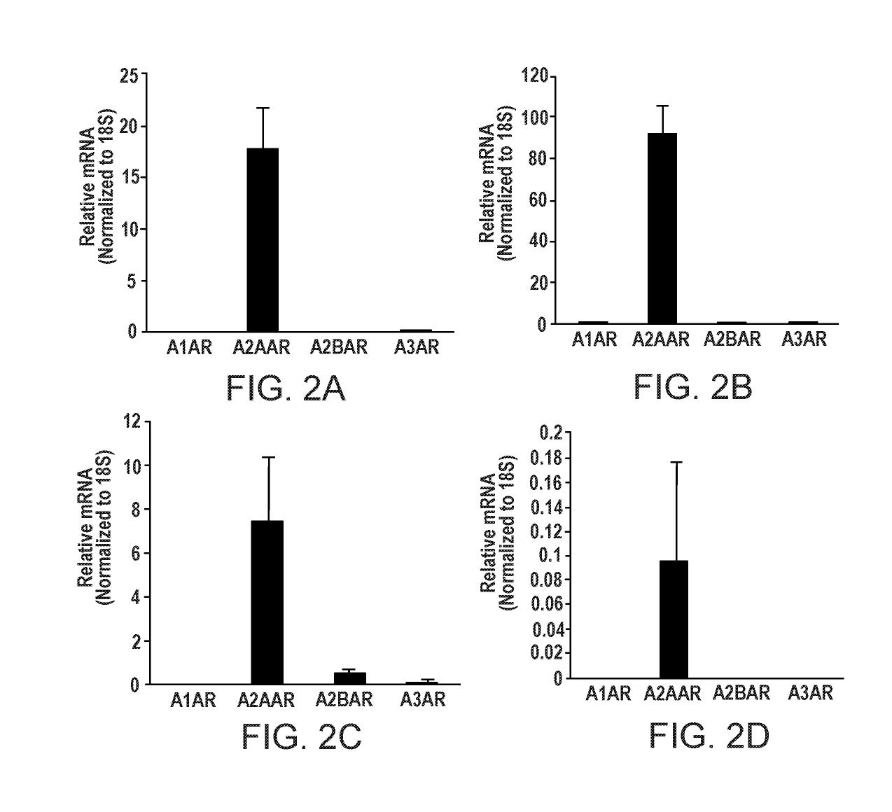

While resting Th cells express low levels of adenosine receptor mRNA (Lappas C M, Rieger J M, Linden J. A_{2A} adenosine receptor induction inhibits IFN-gamma production in murine CD4+ T cells. Journal of Immunology. 2005; 174(2): 1073-80; incorporated herein in its entirety), anti-CD3-activated Th, including Teffector (Teff) and Treg cells (FIG. 2A and FIG. 2B) express the A_{2A}AR almost exclusively as do ILC (FIG. 2C and FIG. 2D). CD4+Th cells were separated into effector Th cells (Teff) (FIG. 2A) or Treg (FIG. 2B) by magnetic beads and fluorescence-activated cell sorting. Innate lymphoid cells type 1 (FIG. 2C) or 3 (FIG. 2D) were purified from spleen (ILC1) or mucosal tissues (ILC3) using antibodies to deplete cell of non ILC lineage using markers for antigen presenting cells (CD11b/c; class II MHC), B cells (B220) and T cells (CD3 or TcRβ). The lineage-cells were positively enriched for NKp46+(ILC1) while ILC3 were selected based on Thy1.2+, CD127⁺ from BL/6 mice or GFP+ lymphoid cells from Roryt-GFP-Rag1 KO mice since ILC3 express Rorγt. Cells were pooled and subsequently, mRNA was extracted and assayed by real-time RT PCR normalized to 18S rRNA CT34 to quantify the relative number of transcripts for the 4 adenosine receptor subtypes. Supporting the notion that the A_{2A}AR is a key target, antigen presenting cells, including macrophages and dendritic cells, (data not shown) and neutrophils (additional data not shown) (Sullivan G W, Rieger J M, Scheld W M, Macdonald T L, Linden J. Cyclic AMP-dependent inhibition of human neutrophil oxidative activity by substituted 2-propynylcyclohexyl adenosine A(2A) receptor agonists. Br J Pharmacol. 2001; 132(5): 1017-26; incorporated herein in its entirety) express the A_{2A}AR subtype which inhibits their pro-inflammatory functions. Human epithelial cells only express the A_{2B}AR (data not shown).

### Example 3: Determining the Role of Adenosine-Mediated Responses to Infection on Persistence

Wildtype (BL/6) or KO mice (e.g. A_{2A}/A_{2B} DKO (A2A/B), A_{2B} KO (A2B), A_{2A} KO (A2A)) or CD73 KO mice were infected with H. pylori and the effect on gastritis and bacterial burden was assessed. Wildtype (BL/6) or A_{2A}/A_{2B} DKO (A2A/B), A_{2B} KO (A2B), A_{2A} KO (A2A) or CD73 KO (CD73) mice were infected by gavage three times (every other day) with 1×10⁸ CFU H. pylori (strain Hp SS1=I) or left uninfected (UI) and housed for 6 weeks. Importantly, the mice were housed in same room in which we have documented their microbiome by sequencing 16S ribosomal DNA to ensure we are aware of any changes in their microbial communities that could affect colonization with H. pylori. Subsequently, the mice were euthanized, used as a source of Th cells and their stomachs were evaluated for gastritis and bacterial burden. The changes due to infection reflected an increase in polymorphonuclear cells, lymphocytes and antigen presenting cells.

The effects of the A_{2B}AR were paradoxical, with low constitutive inflammation and a marked induction after infection. Gastritis was increased in uninfected mice lacking the A_{2A}AR but not in mice lacking the A_{2B}AR (FIG. 3 and FIG. 4). Increases in gastritis scores in the cohorts of KO mice tested ranged from 20% to 500% greater than uninfected control strains of mice (FIG. 3 and FIG. 4). When compared to infected BL/6 mice, the gastritis scores in KO mice ranged from 0% to 20% higher. This increase in gastritis was generally not associated with a significant decrease in bacteria suggesting that antigen-specific responses are required.

Further, Th cells and ILC expressed the A_{2A}AR almost exclusively while antigen presenting cells and neutrophils also express this receptor.

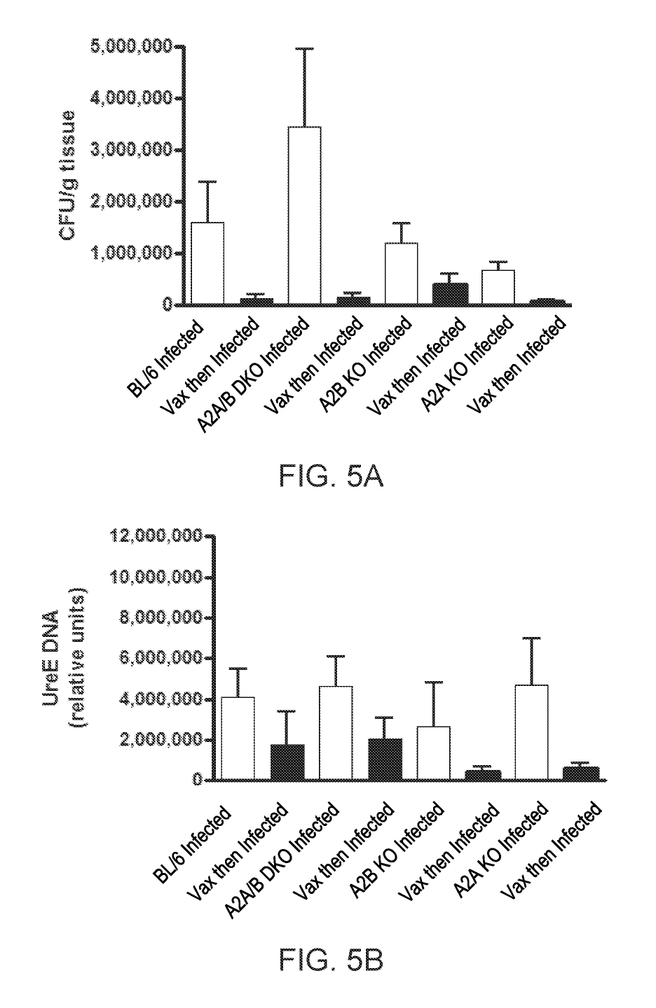

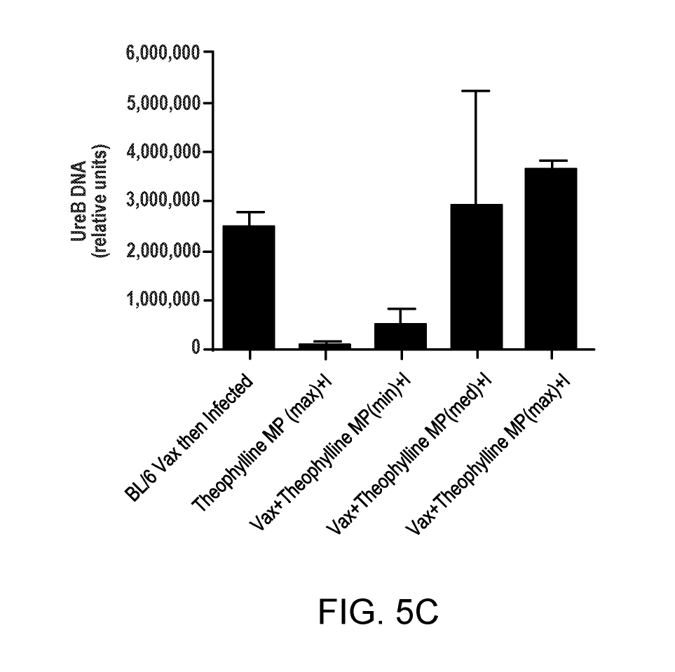

Bacterial burden was assessed by culture (CFU/g tissue) (FIG. 5A) or by PCR (relative units of UreE DNA)(FIG. 5B and FIG. 5C) to quantify H. pylori-specific gene (UreE). The data indicate that the absence of the A_{2A}AR has the most predictable effect on gastritis, and further that the A_{2A}AR is the predominant adenosine receptor expressed by most of the cells believed to be important in immunity to H. pylori.

The A_{2A}AR KO mice tended to have lower bacterial burdens (FIG. 5) again suggesting that it may be the preferred target for pharmacological manipulation of immunity.

### Example 4: Comparison of the Different Adenosine Receptor Subtypes and their Contribution to Immunity

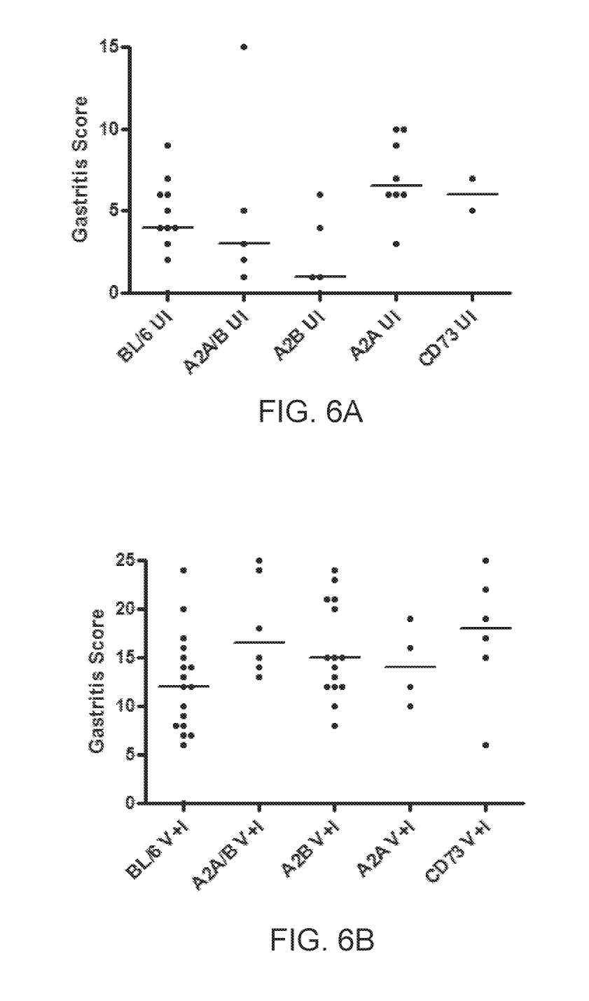

To determine if the absence of adenosine receptor signaling would enhance immunity, wildtype or KO mice (e.g. A_{2A}AR, A_{2B}AR, A_{2A}/_{2B}AR DKO) or CD73 KO mice were immunized, infected with H. pylori and the effect on gastritis and bacterial burden were assessed. Wildtype (BL/6) or A_{2A}/A_{2B} DKO (A2A/B), A_{2B} KO (A2B), A_{2A} KO (A2A) or CD73 KO (CD73) mice were orally immunized (H. pylori sonicate plus cholera toxin) two times, rested for 1 week and then challenged by gavage three times (every other day) with 1×10⁸ CFU H. pylori (SS1=I) (FIG. 6B) or left uninfected (UI) and housed for another 6 weeks (FIG. 6A). Subsequently, the mice were euthanized and evaluated for gastritis or bacterial colonization. The gastritis scores in the cohorts of immunized and infected KO mice (FIG. 6B) ranged from 120% to 300% greater than uninfected control mice (FIG. 6A). Infection or immunization and challenge of all strains of mice doubled the absolute number of Th1 and Th17 cells in gastric lymph nodes although the relative percentage of each Th cell subset did not change. When compared to the immunized and challenged BL/6 mice, the gastritis scores ranged from 0% to approximately 30% higher.

This increase in gastritis was associated with a significant decrease in bacterial burden following immunization (FIG. 5). Moreover, the A_{2A}AR KO mice had the lowest absolute bacterial burden. The immunized A_{2A}AR KO mice had 65% lower bacterial burden than immunized BL/6 mice by CFU and >90% less by PCR. This observation further supports the notion that inhibiting the effect of adenosine on A_{2A}AR increases immunity.

The ability of nanoparticles that release a nonselective adenosine receptor antagonist to enhance immunity when administered with the vaccine was tested.

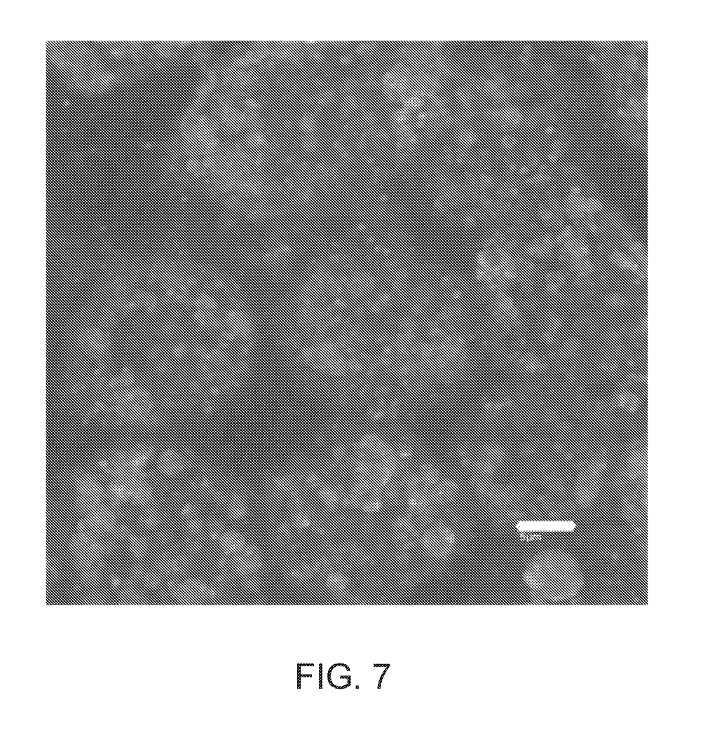

To evaluate the uptake of nanoparticles by macrophage cells, nanoparticles were manufactured by the addition of an organic solution of PLGA 50:50 and red fluorescent dye (DiD) to an aqueous solution containing chitosan and theophylline and then mixed with high speed stirring. The particles were washed by centrifugation in Sterile Water Injection, USP. Uptake, by macrophage cells was determined by fluorescence microscopy. As shown in FIG. 7, the nanoparticles are taken up by antigen presenting cells so when administered with the vaccine, they release the drug and provide a local inhibition of the adenosine receptors.

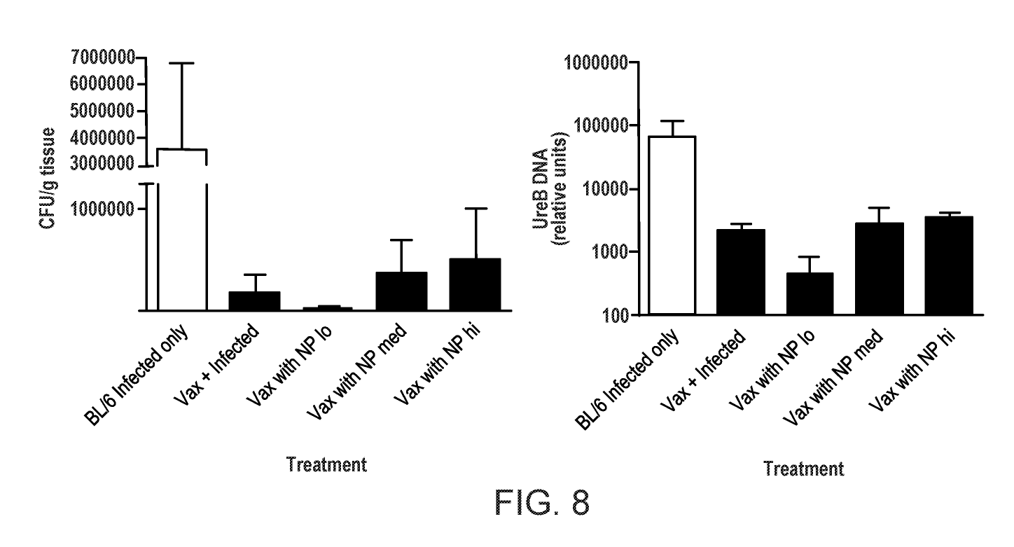

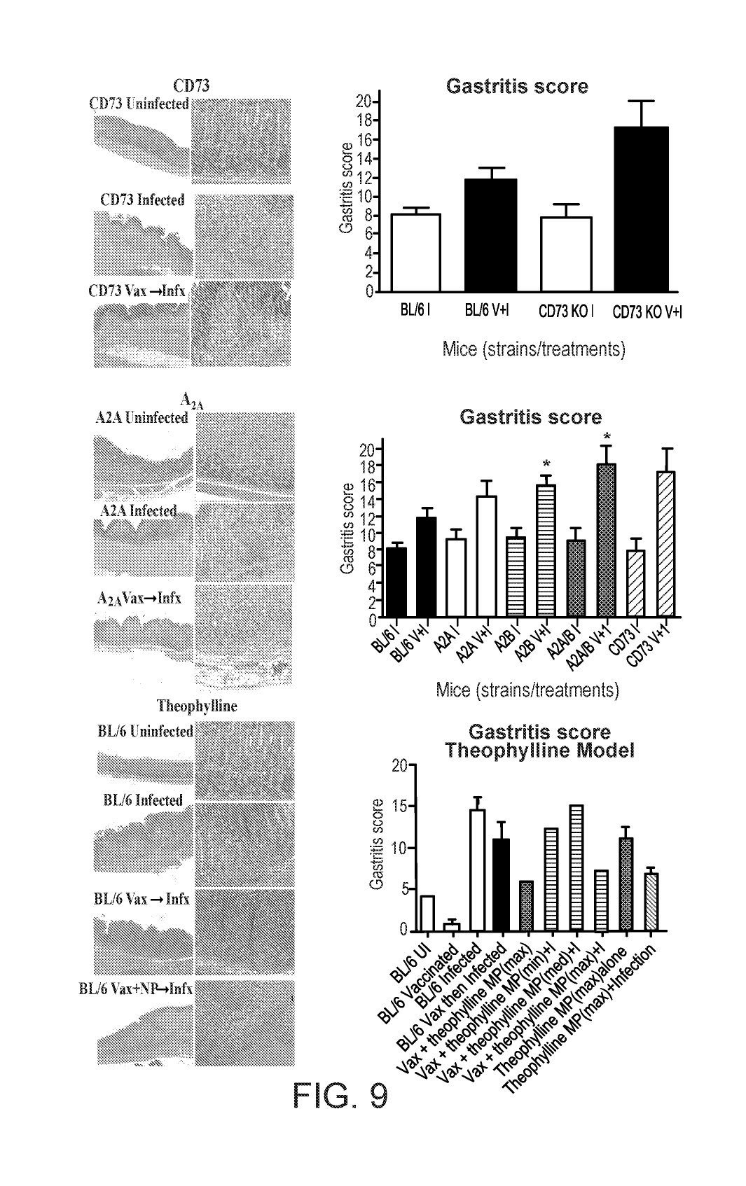

To evaluate the effect of the nanoparticles on immunity, C57BL/6 mice were immunized with or without varying numbers of nanoparticles (low, medium or high dose), challenged with H. pylori and the effect on gastritis and bacterial burden was assessed. Gastritis scores were again increased in the cohorts of BL/6 mice receiving the adenosine receptor antagonist (data not shown). As demonstrated in FIG. 8, immunity was enhanced almost 1 log (i.e. >99% decrease in bacterial burden) in mice receiving the low dose of the nanoparticles releasing theophylline compared to the control mice receiving vaccine alone. The nanoparticles releasing theophylline were administered at a dose of 0.05 nM, 0.5 nM, and 5 nM of the adenosine receptor antagonist (low, medium, and high respectively). Nanoparticles releasing SCH58261 were administered at a 1 pM, 10 pM, and 100 pM of the adenosine receptor antagonist.

When adenosine responsiveness (A_{2A}AR or A_{2B}AR KO mice) were disrupted genetically or pharmacologically (theophylline), immunization led to a substantial decrease in bacteria after challenge. Bacterial burden was approximately 8.5 fold less (21,088 CFU/g tissue vs. 178,639) when vaccine was supplemented with a low dose of nanoparticles loaded with theophylline. Thus, administration of theophylline orally in microparticles at the time of immunization enhanced immunity in BL/6 mice. Theophylline worked best at the lower concentration, possibly due to its side effects of inhibiting phosphodiesterase activity at higher concentrations. At higher doses, the beneficial effect was lost, perhaps due to targeting adenosine receptors with lower affinity that induce competing responses and/or other off target effects. Inhibition of phosphodiesterase would allow cAMP to accumulate in cells—a chemical change that inhibits their pro-inflammatory potential.

The degree of protection was greater in A_{2A} and A_{2B}AR knock-out strains after vaccination compared to the same manipulations of BL/6 controls. The low dose of theophylline was able to boost bacterial clearance in wildtype, BL/6 mice (FIG. 5 and FIG. 8).

The decrease in benefit with the increase in the number of nanoparticles may be due to “off target” effects of the theophylline that impair immunity, or higher concentrations of theophylline may bind lower affinity receptors that favor immunity. We conclude that the effect of the adenosine mediator produced by Treg is on its numerous target cells rather than on Treg alone.

### Example 5: Histological Analysis of Samples with Disrupted Adenosine Function

Following disruption of adenosine production (CD73 KO mice) or responsiveness (either A_{2A}AR KO mice or low dose of theophylline), H. pylori infection induced gastritis characterized by clusters of lymphocytes, plasma cells, and neutrophils infiltrating the lamina propria, and extending into the underlying submucosa. There was mild to moderate parietal cell loss. In vaccinated mice following infection, the inflammatory infiltrate was more pronounced, with a larger component comprised of neutrophils, which expanded extensively into the submucosa. Parietal cell loss was more severe, with occasional replacement by mucous neck cells (mucous cell metaplasia and hyperplasia). Additional findings included dilated gastric glands and glandular abscesses.

The degree of gastritis was greater in all knock-out strains after vaccination or infection compared to the same manipulations of BL/6 controls (FIG. 9). Gastritis was assessed in various strains before or after infection subsequent to immunization. Gastritis was compared in BL/6 mice and mice lacking the ability to synthesis adenosine (CD73 knockout—KO—mice) (see FIG. 9 top panel titled CD73). Mice deficient in the ability to synthesize the anti-inflammatory mediator adenosine had more gastritis (see FIG. 9 top right bar graph). Mice deficient in ability to synthesize adenosine (CD73) were compared to mice lacking adenosine receptors—either the A2A; A2B or both (A2A/B) (FIG. 9 middle panel). Gastritis was also evaluated in mice that were given various concentrations of nanoparticles (NP) containing theophylline along with the vaccine (VAX) and after infection (I) (FIG. 9 bottom panel titled Theophylline). Mice given the intermediate concentration of particles had the highest inflammation scores

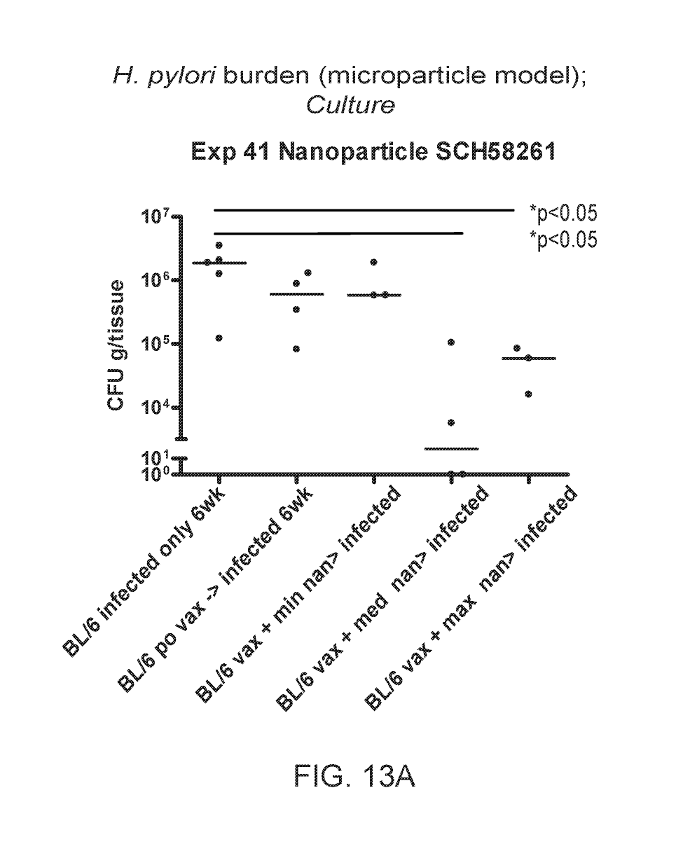

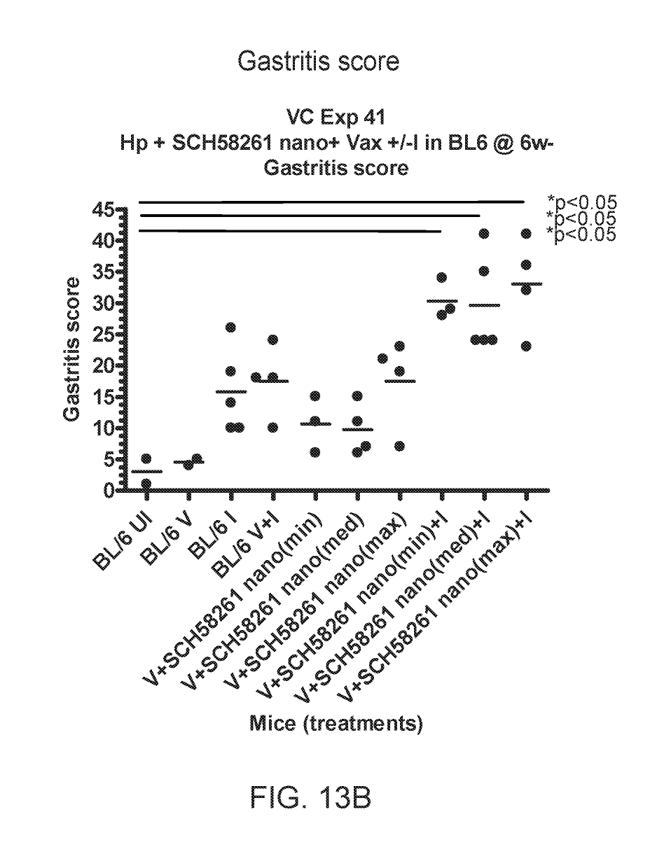

The adjuvant effect of nanoparticles comprising PLGA, SCH58261, and chitosan was then determined (FIG. 13). The A2A adenosine receptor is believed to have the greatest anti-inflammatory effect, so nanoparticles were loaded with a specific A2A antagonist (SCH58261) to enhance gastritis and improve immunity. Mice were immunized with one of three concentrations (min=1 pM, med=10 pM, max=100 pM, SCH58216) of the particles (nano), infected and assessed for bacterial burden (FIG. 13A) or gastritis (FIG. 13B). The mice treated with nanoparticles were compared to BL/6 mice that were infected only or vaccinated and infected. Persistent infection was attenuated or cleared at the medium (med) concentration of nanoparticles. FIG. 13B shows that nanoparticles loaded with the A2A receptor antagonist combined with the vaccine increased gastritis (V+SCH58261) compared to vaccine alone (V). Gastritis reached its maximum in this cohort (FIG. 13B).

### Example 6: Antitumor Adjuvant Effect

The adjuvant properties of a formulation including PLGA, SCH58261 and chitosan were determined by direct injection of the nanoparticles into tumors. Adenosine signaling is difficult to block in tumors as it can reach very high concentrations in a tumor (>1 μM). The A2AR antagonist SCH58261 was encapsulated into PLGA particles (SCH-particles) in order to deliver SCH selectively to phagocytic myeloid cells. The SCH-particles were injected into solid tumors (IT) and were selectively engulfed by tumor associated macrophages and DCs.

The immune response to the intratumoral injection of SCH-particles was then determined by injecting 4T1-1 2B mammary carcinoma cells into the right and left mammary fat pads and one side was treated with SCH-particles. In contrast to IP administration of 1 mg/kg SCH58261, which minimally reduced tumor growth, IT administered SCH-particles (containing 10,000 times less total SCH than the IP injections) were highly effective and eradicated both treated and untreated primary tumors and prevented lung metastases. IT injection of free SCH58261 antagonist was much less effective than injection of SCH-particles. Following SCH-particle injection, there was increased APC activation in the tumor draining lymph node on the treated but not untreated side. SCH-particle treatment reduced numbers of Tregs in tumors and tumor draining lymph nodes on both sides, and increased CTL activation and numbers. SCH-particles targeted tumor infiltrating phagocytic cells, induced systemic immune activation without causing adverse effects on T-cell survival, and produced minimal systemic exposure to the antagonist.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A particle comprising a permeation enhancer and an adenosine receptor antagonist, wherein the particle is a nanoparticle or microparticle.

2. The particle of claim 1, wherein the particle comprises a biodegradable polymer.

3. The particle of claim 1, further comprising an antigen selected from the group consisting of a bacterial antigen, a viral antigen and a tumor antigen.

4.-8. (canceled)

9. The particle of claim 1, wherein the adenosine receptor antagonist is selected from the group consisting of caffeine, theophylline, 8-phenyl theophylline, SCH58261, istradefylline, pyrazolo[4,3-e]-1,2,4-triazolo[1,5-c]pyrimidines or substituted derivatives thereof (e.g., methoxy biaryl or quinoline substitutions), SCH412348, SCH420814, fused heterocyclic pyrazolo[4,3-e]-1,2,4-triazolo[1,5-c]pyrimidines or substituted derivatives thereof (e.g., tetrahydyroisoquinoline or azaisoquinoline derivatives), aryl piperazine substituted 3H-[1,2,4]-triazolo[5,1-i]purin-5-amines, arylindenopyrimidines, arylindenopyrimidines or substituted derivatives thereof, pyrazolo[4,3-e]-1,2,4-trizolo[4,3-c]pyrimidon-3-one and thiazolotriazolopyrimidines, 1,2,4-triazolo[1,5-c]pyrimidines or substituted derivatives thereof, purinones or substituted derivatives thereof, thieno[3,2-d]pyrimidines, pyrazolo[3,4-d]pyrimidines, and 6-arylpurines, benzyl substituted triazolo[4,5-d]pyrimidines, triazolo-9H-purines, aminomethyl substituted thieno[2,3-d]pyrimidines, 2-Aminoimidazopyridines, 4-morpholino-benzothiazoles or substituted derivatives thereof, 4-Aryl and 4-morpholino substituted benzofurans, pyridone substituted pyrazines, heterocyclic substituted 2-amino-thiazoles, tri substituted pyrimidines, piperazine substituted pyrimidine acetamides, acylaminopyrimidines, pyrimidine, pyridine, or triazine carboxamides, and mixtures or and pharmaceutically acceptable salts thereof.

10. The particle of claim 1, wherein the permeation enhancer is selected from the group consisting of chitosan material, a fatty acid, a bile salt, a salt of fusidic acid, a polyoxyethylenesorbitan, a sodium lauryl sulfate, polyoxyethylene-9-lauryl ether (LAURETH™-9), EDTA, citric acid, a salicylate, a caprylic glyceride, a capric glyceride, sodium caprylate, sodium caprate, sodium laurate, sodium glycyrrhetinate, dipotassium glycyrrhizinate, glycyrrhetinic acid hydrogen succinate, a disodium salt, a nacylcarnitine, a cyclodextrin, a phospholipid, and mixtures thereof.

11. The particle of claim 2, wherein the biodegradable polymer is selected from the group consisting of a polyester, a lactic acid polymer, copolymers of lactic acid and of glycolic acid, poly-ε-caprolactone (PCL), poly(anhydrides), poly(amides), poly(urethanes), poly(carbonates), poly(acetals), poly(ortho-esters), poly(glycolide-co-trimethylene carbonate), poly(dioxanone), poly(phosphoesters), poly(phosphazenes), poly(cyanoacrylate), poly(ethylene oxide), poly(propylene oxide), poly(N-isopropylacrylamide) (PNIPAAm), poly(2-(diethylamino)ethyl methacrylate) (PDEAEMA), poly(2-aminoethyl methacrylate) (PAEMA), 2 (dimethylamino)ethyl methacrylate (DMAEMA), poly(ethylene glycol) (PEG), N-(2-hydroxypropyl)methacrylamide (HPMA), poly(β-benzyl-1-aspartate) (PBLA), poly(hydroxybutyrate-co valerate), and mixtures or derivatives thereof.

12.-14. (canceled)

15. The particle of claim 1, wherein the particle has an average diameter of about 0.5 nm to about 80 μm.

16.-17. (canceled)

18. The particle of claim 2, wherein the biodegradable polymer is PLGA, the permeation enhancer is chitosan, and the adenosine receptor antagonist is selected from the group consisting of SCH58261 and theophylline.

19. The particle of claim 1, further comprising a targeting moiety.

20.-21. (canceled)

22. A pharmaceutical composition comprising the particle of claim 1.

23.-24. (canceled)

25. A vaccine composition comprising:
a particle comprising a permeation enhancer and an adenosine receptor antagonist; and
an antigen.

26.-29. (canceled)

30. The vaccine composition claim 25, further comprising a therapeutic agent selected from the group consisting of an antimicrobial agent, an antibiotic agent, an anti-fungal agent, an anti-cancer agent, an anti-tumor agent, a signaling protein, a small molecule drug, a nucleic acid composition, a peptide therapeutic and an antibody.

31.-40. (canceled)

41. The vaccine composition of claim 25, further comprising a targeting moiety.

42.-43. (canceled)

44. The vaccine composition of claim 25, wherein the vaccine is formulated for parenteral, intravenous, intradermal, subcutaneous, oral, inhalation, transdermal, transmucosal, rectal and intratumoral administration.

45. A method of treating an infectious disease, comprising administering a therapeutically effective amount of the pharmaceutical composition of claim 22.

46.-53. (canceled)

54. A method of treating a tumor in a subject in need thereof, comprising administering to the subject a therapeutically effective amount of the pharmaceutical composition of claim 22.

55. The method of claim 54, wherein the pharmaceutical composition comprises an anti-tumor antigen.

56.-62. (canceled)

63. A method of treating a H. pylori infection in a subject in need thereof, comprising administering to the subject a therapeutically effective amount of the pharmaceutical composition of claim 22 to the subject.

64.-69. (canceled)

70. A method of enhancing an immune response to an antigen comprising:
administering:
a particle comprising
a biodegradable polymer;
a permeation enhancer; and
an adenosine receptor antagonist; and
an antigen.

71.-79. (canceled)

80. An adjuvant composition comprising a particle, the particle comprising:
a biodegradable polymer,
an permeation enhancer, and
an adenosine receptor antagonist,
wherein the particle is a nanoparticle or microparticle.

81.-83. (canceled)

84. The adjuvant composition of claim 80, further comprising an antigen.

85. The adjuvant composition of claim 84, wherein the antigen is a disease associated protein selected from beta amyloid proteins, tau, prion proteins or its fragments, alpha-synuclein, superoxide dismutase 1, Huntingtin fragments, transthyretin, beta2-microglobulin, Apo A-1 fragments, Apo-AII, Apo AIV, TDP-43, FUS, ABri, Adan, crystallins, calcitonin, atrial natriuretic facto, prolactin, keratins, Cyrstatin C, Notch3, Glial fibrillary acidic protein (GFAP), seipin, cystic fibrosis transmembrane conductance regulator (CFTR) protein, and amylin.
